(19) United States
(12) Patent Application Publication
(10) Pub. No.: US 2022/0280607 A1
(43) Pub. Date: Sep. 8, 2022

(54) Treatment of Heart Defects and Conditions in Pediatric Patients

(71) Applicant: University of Pittsburgh - Of the Commonwealth System of Higher Education, Pittsburgh, PA (US)

(72) Inventors: Bernhard Kuhn, Pittsburgh, PA (US); Honghai Liu, Pittsburgh, PA (US)

(21) Appl. No.: 17/625,270

(22) PCT Filed: Jul. 13, 2020
(86) PCT No.: PCT/US2020/041808

§ 371 Date: Jan. 6, 2022

Related U.S. Application Data
(60) Provisional application No. 62/873,483, filed on Jul. 12, 2019.

(51) Int. Cl. A61K 38/18; A61K 31/138; A61K 38/17; A61P 9/00; A61K 45/06

(52) U.S. Cl. CPC A61K 38/1825; A61K 31/138; A61K 38/1709; A61K 38/1883; A61P 9/00; A61K 45/06

(57) ABSTRACT

Provided herein are methods of treating congenital heart disease in a patient, such as tetralogy of Fallot, with beta blockers to increase cardiomyocyte endowment in the patient and/or to reduce risk of developing complications originating later from heart diseases, such as myocardial infarction, in the patient. Also provided herein are uses for beta blockers for treating congenital heart disease, such as tetralogy of Fallot, in a patient to increase cardiomyocyte endowment in the patient and/or to reduce risk of developing complications originating later from heart diseases, such as myocardial infarction, in the patient.

### CROSS REFERENCE TO RELATED APPLICATIONS

This application claims the benefit of U.S. Provisional Patent Application No. 62/873,483 filed Jul. 12, 2019, the disclosure of which is incorporated herein by reference in its entirety.

### STATEMENT REGARDING FEDERALLY SPONSORED RESEARCH

This invention was made with government support under Grant Nos. HL106302 and TR001857 awarded by the National Institutes of Health. The government has certain rights in the invention.

### REFERENCE TO A SEQUENCE LISTING SUBMITTED VIA EFS-WEB

The content of the ASCII text file of the sequence listing named “6527_2001910_ST25” which is 15,112 bytes in size was created on Jul. 13, 2020 and electronically submitted herewith via EFS-Web, and is hereby incorporated by reference in its entirety.

Provided herein are methods for treating patients with heart defects, which may be associated with low cardiomyocyte endowment (lower than normal number of cardiomyocytes), such as patients having Tetralogy of Fallot. Beta-blocker are used to counter cytokinesis failure in pediatric patients, thereby preventing ventricular remodeling, heart failure, and arrhythmia development.

Congenital heart disease (CHD) is the most common birth defect. CHD occurs in ˜1% of live births in the US, with similar prevalence throughout the world. Improvements in diagnosis and treatment have increased survival rates, enabling one million patients to live with CHD in the US. Many forms of CHD have right ventricular (RV) hypertension. In Tetralogy of Fallot with pulmonary stenosis (ToF/PS), the most common form of cyanotic CHD and the form most available for research studies, RV hypertension is due to outflow tract obstruction. Patients with ToF/PS have a high lifetime risk of developing RV failure and arrhythmias.

Even after successful surgical repair, patients with CHD are at a significantly increased risk for heart failure and arrhythmia, leading to premature death, for their lifetime. As a result, although current therapies have extended life expectancy for patients with CHD, CHD remain an enormous burden on the economy. Hospital costs for patients with CHD exceeded $5.6 billion in 2009 (source: CDC). Although patients with CHD comprised only 3.7% of total hospitalizations, associated costs were 15.1% of the total costs for all US hospitalizations for children and adolescents aged 0-20 years. Major hemodynamic abnormalities before and after CHD surgery increase the workload of the heart, and it is thought that these are major contributing factors to the increased lifetime risk of death from heart failure and arrhythmia. However, besides surgery to decrease the hemodynamic load, no effective therapies are available.

Effective treatments for CHD are needed.

### SUMMARY

A method of treating patients less than 6 months past term having a congenital heart defect and a reduced (low) cardiomyocyte endowment resulting from heart cell division failure (e.g., cytokinesis failure) is provided The method comprises administering to the patient a nonspecific beta-blocker, a β1-beta blocker, a β2-beta blocker, or a combination thereof, in an amount and for a duration effective to induce successful cardiomyocyte cytokinesis in the patient and expansion of the cardiomyocyte endowment in the patient, thereby reducing a percentage of binucleated cells in heart tissue in the patient, increasing cardiomyocyte endowment by at least 5% in the patient, and/or improving heart function and resilience to heart injury, such as myocardial infarction in the patient.

Also provided herein is a beta blocker, such as a nonspecific beta-blocker, a β₁-beta blocker, a β₂-beta blocker, or a combination thereof, for use in treatment of a patient less than 6 months past term having a congenital heart defect resulting from reduced cardiomyocyte endowment resulting from cytokinesis failure, wherein the beta blocker is administered to the patient in an amount and for a duration effective to induce cardiomyocyte cytokinesis in the patient and expansion of the cardiomyocyte endowment in the patient, thereby reducing a percentage of binucleated cells in heart tissue in the patient, and/or increasing cardiomyocyte endowment by at least 5% in the patient.

The following numbered clauses describe various embodiments, aspects, and/or examples of the present invention.

Clause 1. A method of treating patients less than 6 months past term having a congenital heart defect and a reduced (low) cardiomyocyte endowment resulting from heart cell division failure (e.g., cytokinesis failure, comprising administering to the patient a nonspecific beta-blocker, a β₁-beta blocker, a β₂-beta blocker, or a combination thereof, in an amount and for a duration effective to induce successful cardiomyocyte cytokinesis in the patient and expansion of the cardiomyocyte endowment in the patient, thereby reducing a percentage of binucleated cells in heart tissue in the patient, increasing cardiomyocyte endowment by at least 5% in the patient, and/or improving heart function and resilience to heart injury, such as myocardial infarction in the patient.

Clause 2. The method of clause 1, further comprising determining a percentage of binucleated cardiomyocytes in heart tissue of the patient, determining a presence of multinucleated cardiomyocytes in heart tissue of the patient at one or more times prior to or during administration of the beta-blocker to the patient.

Clause 3. The method of clause 2, further comprising determining a percentage of binucleated cardiomyocytes in heart tissue of the patient at two or more time points including a time point during or after administration of the beta blocker to the patient, and determining if the percentage of binucleated cardiomyocytes in the heart tissue is decreased, indicating expansion of the cardiomyocyte endowment in the patient.

Clause 4. The method of any one of clauses 1-3, comprising determining heart tissue growth or cardiac mass in the patient to determine an increase in cardiomyocyte endowment in the patient.

Clause 5. The method of any one of clauses 1-3, wherein the congenital heart defect results in above normal RVSP, further comprising determining right ventricle systolic pressure (RVSP) at one or more time points during treatment of the patient with the beta blocker.

Clause 6. The method of any one of clauses 1-5, comprising discontinuing administration of the beta blocker after determining that the binucleated cardiomyocyte percentage in heart tissue in the patient is normalized and/or cardiomyocyte endowment is increased at least 5% in the patient.

Clause 7. The method of any one of clauses 1-6, wherein the patient is non-cyanotic or non-hypoxic.

Clause 8. The method of any one of clauses 1-7, wherein the beta blocker is a nonspecific beta-blocker.

Clause 9. The method of any one of clauses 1-7 wherein the beta blocker is a β₂ beta-blocker.

Clause 10. The method of any one of clauses 1-7, wherein the beta-blocker comprises propranolol or alprenolol.

Clause 11. The method of any one of clauses 1-10, wherein the congenital heart defect is a defect associated with tetralogy of Fallot.

Clause 12. The method of any one of clauses 1-11, wherein the patient has a hypoplastic or absent conal septum, stenosis of the left pulmonary artery, a bicuspid pulmonary valve, a right-sided aortic arch, coronary artery anomalies, a patent foramen ovale or atrial septal defect, an atrioventricular septal defect, a partial or complete pulmonary vein return anomaly, and/or pulmonary atresa.

Clause 13. The method of any one of clauses 1-10, wherein the congenital heart defect is, or is a defect associated with: trilogy of Fallot; aortic valve stenosis; coarctation of the aorta; Ebstein's anomaly; patent ductus arteriosus; pulmonary valve stenosis; septal defect, such as an atrial septal defect or an ventricular septal defect; a single ventricle defect, such as hypoplastic left heart syndrome or tricuspid atresia; total or partial anomalous pulmonary venous connection (TAPVC); transposition of the great arteries; or truncus arteriosus.

Clause 14. The method of any one of clauses 1-10, wherein the congenital heart defect is an anterior malalignment of the infundibular septum with the muscular septum.

Clause 15. The method of any one of clauses 1-12, wherein the congenital heart defect is one or more of pulmonary valve stenosis, a ventricular septal defect, an overriding aorta, and right ventricular hypertrophy.

Clause 16. The method of any one of clauses 1-15, wherein the patient has undergone surgery to repair one or more defects resulting from the congenital heart disease in the patient, and the beta blocker is administered to the patient continuously for at least two weeks, or for at least one month after the surgery to increase cardiomyocyte endowment in the patient.

Clause 17. The method of any one of clauses 1-16, wherein treatment of the patient with the beta blocker is initiated prior to closure of the foramen ovale in a patient not having a patent foramen ovale or ductus arteriosus, in a patient not having patent ductus arteriosus.

Clause 18. The method of any one of clauses 1-17, wherein the patient is human.

Clause 19. The method of any one of clauses 1-18, to lower risk of complications relating to myocardial infarction in the patient, such as heart failure.

Clause 20. The method of any one of clauses 1-19, further comprising administering one or more additional therapeutic agents to the patient during treatment of the patient with the beta blocker.

Clause 21. The method of clause 20, wherein the one or more additional therapeutic agents is a cml growth factor or mitogen in an amount effective to stimulate cardiomyocyte cell growth or expansion in the patient.

Clause 22. The method of clause 21, wherein the cell growth factor or mitogen is periostin, neuregulin, or a fibroblast growth factor.

Clause 23. The method of clause 21, wherein the cell growth factor or mitogen is NRG61 (SEQ ID NO: 2, e.g., NEUCARDIN™).

Clause 24. A beta blocker, such as a nonspecific beta-blocker, a β₁-beta blocker, a β₂-beta blocker, or a combination thereof, for use in treatment of a patient less than 6 months past term having a congenital heart defect resulting from reduced cardlomyocyte endowment resulting from cytokinesis failure, wherein the beta blocker is administered to the patient in an amount and for a duration effective to induce cardiomyocyte cytokinesis in the patient and expansion of the cardiomyocyte endowment in the patient, thereby reducing a percentage of binucleated cells in heart tissue in the patient, and/or increasing cardiomyocyte endowment by at least 5% in the patient.

Clause 25. The beta blocker for use of clause 24, wherein a percentage of binucleated cardiomyocytes in heart tissue of the patient, or a presence of multinucleated cardiomyocytes is determined in heart tissue of the patient at one or more times prior to or during administration of the beta-blocker to the patient.

Clause 26. The beta blocker for use of clause 25, wherein a percentage of binucleated cardiomyocytes in heart tissue of the patient is determined at two or more time points including a time point during or after administration of the beta blocker to the patient, and whether the percentage of binucleated cardiomyocytes in the heart tissue is decreased is determined, indicating expansion of the cardiomyocyte endowment in the patient.

Clause 27. The beta blocker for use of any one of clauses 24-26, wherein heart tissue growth or cardiac mass in the patient is determined to determine a degree of repair of the one or more heart defects resulting from reduced cardiomyocyte endowment or to determine an increase in cardiomyocyte endowment in the patient.

Clause 28. The beta blocker for use of any one of clauses 24-27, wherein the congenital heart defect results in above normal right ventricle systolic pressure (RVSP), and wherein RVSP is determined one or more times during treatment of the patient with the beta blocker.

Clause 29. The beta blocker for use of any one of clauses 24-28, wherein administration of the beta blocker is discontinued after determining that the binucleated cardiomyocyte percentage in heart tissue in the patient is normalized and/or cardiomyocyte endowment is increased at least 5% in the patient.

Clause 30. The beta blocker for use of any one of clauses 24-29, wherein the patient is non-cyanotic or non-hypoxic.

Clause 31. The beta blocker for use of anyone of clauses 24-30, wherein the beta blocker is a nonspecific beta-blocker.

Clause 32. The beta blocker for use of any one of clauses 24-30, wherein the beta blocker is a β₂ beta-blocker.

Clause 33. The beta blocker for use of any one of clauses 24-30, wherein the beta-blocker comprises propranolol or alprenolol.

Clause 34. The beta blocker for use of any one of clauses 24-33, wherein the congenital heart defect is a defect associated with tetralogy of Fallot.

Clause 35. The beta blocker for use of any one of clauses 24-34, wherein the patient has a hypoplastic or absent conal septum, stenosis of the left pulmonary artery, a bicuspid pulmonary valve, a right-sided aortic arch, coronary artery anomalies, a patent foramen ovale or atrial septal defect, an atrioventricular septal defect, a partial or complete pulmonary vein return anomaly, and/or pulmonary atresia.

Clause 36. The beta blocker for use of any one of clauses 24-33, wherein the congenital heart defect is, or is a defect associated with: trilogy of Fallot; aortic valve stenosis; coarctation of the aorta; Ebstein's anomaly; patent ductus arteriosus; pulmonary valve stenosis; septal defect, such as an atrial septal defect or an ventricular septal defect a single ventricle defect, such as hypoplastic left heart syndrome or tricuspid atresia; total or partial anomalous pulmonary venous connection (TAPVC); transposition of the great arteries; or truncus arteriosus.

Clause 37. The beta blocker for use of any one of clauses 24-33, wherein the congenital heart defect is an anterior malalignment of the infundibular septum with the muscular septum.

Clause 38. The beta blocker for use of any one of clauses 24-35, wherein the congenital heart defect is one or more of pulmonary valve stenosis, a ventricular septal defect, an overriding aorta, and right ventricular hypertrophy.

Clause 39. The beta blocker for use of any one of clauses 21-38, wherein the patient has undergone surgery to repair one or more defects resulting from the congenital heart disease in the patient, and the beta blocker is administered to the patient continuously for at least two weeks, or for at least one month after the surgery to increase cardiomyocyte endowment in the patient.

Clause 40. The beta blocker for use of any one of clauses 24-39, wherein treatment of the patient with the beta blocker is initiated prior to closure of the foramen ovale in a patient not having a patent foramen ovale or ductus arteriosus, in a patient not having patent ductus artenosus.

Clause 41. The beta blocker for use of anyone of clauses 24-40, wherein the patient is human.

Clause 42. The beta blocker for use of anyone of clauses 24-41, for reducing risk of complications associated with myocardial infarction in the patient, such as heart failure.

Clause 43. The beta blocker for use of any one of clauses 24-42, wherein one or more additional therapeutic agents to the patient during treatment of the patient with the beta blocker.

Clause 44. The beta blocker for use of clause 43, wherein the one or more additional therapeutic agents is a cell growth factor or mitogen in an amount effective to stimulate cardiomyocyte cell growth or expansion in the patient.

Clause 45. The beta blocker for use of clause 44, wherein the cell growth factor or mitogen is periostin, neuregulin, or a fibroblast growth factor.

Clause 46. The beta blocker for use of clause 44, wherein the cell growth factor or mitogen is NRG61 (SEQ ID NO: 2, e.g., NEUCARDIN™).

Clause 47. Use of a beta blocker, such as a nonspecific beta-blocker, a β₁-beta blocker, a β₂-beta blocker, or a combination thereof, optionally in combination with a cell growth factor or mitogen such as periostin, neuregulin, or a fibroblast growth factor, for treatment of a congenital heart defect as described in any one of clauses 1-46.

### BRIEF DESCRIPTION OF THE DRAWINGS

FIG. 1 provides an exemplary and non-limiting periostin amino acid sequence (SEQ ID NO: 7).

FIGS. 2A-2E. Quantification (FIG. 2A, FIG. 2B) of cardiomyocytes in heart tissue from patients with tetralogy of Fallot and pulmonary stenosis (ToF/PS), as confirmed by immunostaining, show that cardiomyocytes in infants with ToF/PS fail to divide. Multinucleated (≥2) cardiomyocytes are indicated by filled symbols and solid lines in FIGS. 2A and 2B. Each symbol in FIG. 2A and bar in FIG. 2B represents one human heart (ToF/PS: n=12; No heart disease: n=5). Quantification (FIG. 2C) of ploidy of nuclei in mononucleated cardiomyocytes determined with microscopy and immunostaining (ToF/PS: n=5). Quantification in FIG. 1C includes age-matched published results for no heart disease (n=5) (Mollova et al.). Myocardium were analyzed from a 4-week-old human infant with ToF/PS labeled with ¹⁵N-thymidine (given orally) in FIGS. 2D and 2E. FIG. 2D shows the quantification of labeled binucleated and mononucleated cardiomyocytes (Mononucleated cardiomyocytes analyzed: n=282; ¹⁵N+ mononucleated cardiomyocytes: n=25; Binucleated cardiomyocytes analyzed: n=104 (208 total nuclei); ¹⁵N+binucleated cardiomyocytes: n=20 (40 total nuclei)). The ploidy of ¹⁵N-thymidine positive mononucleated cardiomyocytes was analyzed by microscopy of Hoechst staining of adjacent sections. FIG. 2E shows the quantification of diploid and polyploid cardiomyocytes. Statistical significance was tested with Student's t-test in FIG. 2C.

FIGS. 3A-3H: Ect2 regulates cardiomyocyte cytokinesis and binucleation. Live cell imaging of neonatal rat cardiomyocytes (NRVM, P2-P3, n=52 cardiomyocytes) (FIG. 3A). Cleavage furrow (white arrows) ingression is between 300-335 min, regression at 355 min, and formation of a binucleated cardiomyocyte at 510 min. Transcriptional profiling of single cycling (+) and not cycling (−) cardiomyocytes at embryonic day 14.5 (E14.5) and 5 days after birth (PS) for 61 Dbl-homology family RhoGEFs. Ect2 is significantly repressed in cycling P5 cardiomyocytes (P<0.05) (FIG. 3B). The grayscale intensity code is provided and the frequency of genes with corresponding expression is indicated with a black line. Quantification of RhoA-GTP at the cleavage furrow (E14.5: n=3 cell isolations; P2: n=4 cell isolations) in binucleating cardiomyocytes, as confirmed by immunostaining (FIG. 3C). NRVM transduced with Adv-GFP-Ect2 or Adv-GFP (FIGS. 3D-3H). Live cell imaging of GFP-ECT2 in cycling NRVM (FIG. 3D). Quantification of binucleated cardiomyocytes (n=3 cell isolations) are depicted in FIG. 3E. Quantification of cardiomyocytes in S-phase (n=3 cell isolations) are depicted in FIG. 3F. Quantification of cardiomyocytes in M-phase (n=3 cell isolations) (FIG. 3G). Analysis of ploidy of nuclei (GFP: n=3 cell isolations, GFP-Ect2: n=2 cell isolations) (FIG. 3H). Statistical significance was tested with Student's t-test.

FIG. 4: A single-cell transcriptional profiling strategy Identifies molecular mechanisms of cardiomyocyte binucleation. When neonatal mouse cardiomyocytes are in the cell cycle, two outcomes are possible: the cardiomyocyte may either divide or become binucleated. As such, bulk RNAseq of cycling neonatal mouse cardiomyocytes would not reveal the molecular mechanisms that are specific for binucleation. To overcome this challenge, we have taken a single cell transcriptional profiling approach, which should isolate the specific molecular mechanism. We identified cycling cardiomyocytes with the Azami green-Geminin (mAG-hGem) reporter and isolated single cycling and not cycling cardiomyocytes with FACS.

FIG. 5: The expression of Ect2 gene decreases in cycling neonatal mouse cardiomyocytes. The cycling cardiomyocytes were collected from embryonic (E14.5) and neonatal (P5) from genetic mice that express mAG-hGem as the reporter of cell cycle. The collected cycling cardiomyocytes were then analyzed by single-cell transcriptional profiling. The mRNA expression of the critical cytokinesis genes from the cycling (Gem+) and not cycling (Gem−) are presented above. Mean±SEM are indicated.

FIG. 6: Design of the apoptosis assay. Adenoviral-mediated transduction of GFP-Ect2 does not induce apoptosis in cultured neonatal rat ventricular cardiomyocytes (NRVMs). NRVMs were cultured for 1 day, then Ect2 was expressed in the cells through adenoviral-mediated transduction (Adv-GFP-Ect2, MOI=2,000). Transduction of GFP (MOI=500) was used as negative control. As a positive control for induction of apoptosis, the NRVMs were treated with doxorubicin (1 μM, 24 hours) to induce apoptosis. Apoptosis was detected with the ApopTag Red In Situ apoptosis detection kit (EMD Millipore Corporation), using Doxorubicin (1 μM) as a positive control.

FIGS. 7A-7C: Survival analysis shows that Ect2 gene inactivation in development induces decreased pup viability. To decrease the cardiomyocyte endowment, we inactivated Ect2^{flox} with αMHC-Cre in embryonic mouse hearts. Western blot (FIG. 7A) of Ect2 in αMHC-Cre⁺;Ect2^{F/F} mice at E16.5 (Ect2^{F/Wt} n=3 hearts, Ect2^{F/F} n=3 hearts) (FIG. 7B). Table of genotypes and corresponding condition and number of pups recovered are listed. P-values were calculated using Fisher's exact test (FIG. 7C). Abbreviations used: F/F, flox/flox; F/Wt, flox/+.

FIGS. 8A-8K: Ect2 gene inactivation lowers cardiomyocyte endowment and is lethal in mice (FIGS. 8A-8H). Ect2^{flox} gene inactivation with αMHC-Cre in mice. Quantification of binucleated cardiomyocytes at P1 (Ect2^{F/Wt} n=6, Ect2^{F/F} n=6 hearts), as confirmed by immunostaining (FIG. 8A). DNA content per nucleus (Ect2^{F/Wt} n=642 cardiomyocytes, Ect2^{F/F} n=647 cardiomyocytes) (FIG. 8B). Cardiomyocyte endowment, quantified by counting of fixation-digested hearts (Ect2^{F/Wt} n=12, Ect2^{F/F} n=5 hearts) (FIG. 8C). Quantification of hypertrophy (cardiomyocyte size), as confirmed by immunostaining (FIG. 8D). Quantification of mono- and binudeated cardiomyocyte size (FIG. 8E). In FIGS. 8D-8E, Ect2^{F/Wt} n=1,138 cardiomyocytes, 1101 Mono, 37 Bi, from 6 hearts; Ect2^{F/F} n=1,015 cardiomyocytes, 892 Mono, 123 Bi, from 6 hearts. Heart weight (Ect2^{F/Wt} n=14, Ect2^{F/F} n=6 hearts) (FIG. 8F). Echocardiographic analysis of myocardial dysfunction at P0 (left ventricular endocardium outlined in yellow, Ect2^{Wt/Wt} n=4, Ect2^{F/F} n=3 mice) (FIG. 8G). Pup survival of FIG. 7C (FIG. 8H). Quantification of cardiomyocyte binucleation after Ect2 rescue, as confirmed by immunostaining (n=3 cell isolations) (FIG. 8I). Quantification of cell cycle entry in cardiomyocytes after Ect2^{flox} gene inactivation with αMHC-MerCreMer, tamoxifen P0, P1, P2, followed by 3 days culture in the presence of BrdU, as confirmed by immunostaining (Ect2^{Wt/Wt} n=3, Ect2^{F/F} n=2 cell isolations) (FIG. 8J). Quantification of cell cycle progression to M-phase in vivo after αMHC-Cre inactivation of Ect2^{flox}, as confirmed by immunostaining (P1, Ect2^{F/Wt} n=6, Ect2^{F/F} n=6 hearts) (FIG. 8K). Statistical significance tested with Student's t-test (FIGS. 8A-8D, 8F-8G, 8I-8K), one-way ANOVA with Bonferroni's multiple comparisons (FIG. 8E), and Fisher's exact test (FIG. 8H).

FIG. 9: Inactivation of Ect2 gene in development does not induce apoptosis. The Ect2^{flox} gene in the mouse line αMHC-MerCreMer; Ect2^{flox} was inactivated through intraperitoneal (i.p.) injection of tamoxifen (30 μg/g body weight) to pregnant dams on E14.5, 15.5, and 16.5. Pups were resected and analyzed at E19.5. There is no evidence for cardiomyocyte apoptosis detected by TUNEL assay at E19.5.

FIGS. 10A-10F: Hippo signaling regulates cardiomyocyte abscission and binucleation. Luciferase assay in HEK293 cells analyzing Ect2 promoter activity after removal of the five TEAD1/2-binding sites. WT: wild type Ect2 promoter; Δ1-5: All five putative TEAD-binding sites removed; Δ2 kB: the continuous 2 kB DNA sequence containing all five TEAD-binding sites removed; Vector: Empty vector that did not contain Ect2 promoter (n=3 cultures) (FIG. 10A). Quantification of Ect2 expression after knockdown of TEAD1 and TEAD2 by siRNA (n=4 cell isolations) (FIG. 10B). Quantification of the proportion of binucleated NRVMs after knockdown of TEAD1 and TEAD2, as confirmed by immunostaining (P2, C, n=3 cell isolations) (FIG. 10C). Quantification of binucleated cardiomyocytes generated in NRVM after increasing expression of TEAD1, as confirmed by immunostaining (n=3 cell isolations) (FIG. 10D). Quantification of Ect2 (FIG. 10E) and quantification of binucleated cardiomyocytes after adenoviral overexpression of wild type YAP1 (YAP1-WT) and a non-phosphorylatable version containing a S127A mutation (YAP1-S127A) in NRVMs (P2), as confirmed by immunostaining (n=4 cardiomyocyte isolations) (FIG. 10F). Statistical significance was tested with one-way ANOVA with Bonferroni's multiple comparisons (FIGS. 10A-10C, 10E-10F) and Student's t-test (FIG. 10D).

FIGS. 11A-11G: β-adrenergic receptor signaling regulates cardiomyocyte abscission and binucleation. Cardiac expression of YAP target genes Cyr61 (FIG. 11A) and CTGF (FIG. 11B) and of Ect2 (n=3 hearts/group) after inactivation of β₁- and P2-adrenergic receptor genes (DKO) in mice (P4) (FIG. 11C). Quantification of multinucleated (P4: n=4 hearts/group; P10: n=6 hearts for wild type, n=3 hearts for DKO), as confirmed by immunostaning (FIG. 11D) and total cardiomyocytes in DKO mice (quantified by counting fixation-digested hearts, P4: n=7 hearts for wild type, n=5 hearts for DKO; P10: n=6 hearts for wild type, n=3 hearts for DKO) (FIG. 11E). Quantification of M-phase cardiomyocytes, as confirmed by immunostaining (n=4 hearts/group) in vivo at P4 (FIG. 11F). Quantification of binudeated cardiomyocytes generated by knockdown of Ect2 in cultured neonatal cardiomyocytes from β-AR DKO mice, as confirmed by immunostaining (P2, n=3 cell isolations) (FIG. 11G). Sc: scrambled siRNA. See FIGS. 15A-15E for Ect2 siRNAs validations. Statistical significance was tested with two-way ANOVA with Bonferroni's multiple comparisons (FIGS. 11D-11E) and Student's t-test (FIGS. 11A-11C, 11F-11G).

FIGS. 12A-12H: Pharmacologic alterations of β-adrenergic receptor signaling regulate cardiomyocyte abscission and endowment. Ect2 mRNA expression in cultured NRVMs treated with Forskolin (n=5 cardiomyocyte isolations) (FIG. 12A). Quantification of multinucleated cardiomyocytes after Forskolin administration in vivo, as confirmed by immunostaining (1 μg/g body, 1 i.p. injection per day in newborn mice, n=6 hearts/group) (FIG. 12B). Quantification of multinucleated cardiomyocytes, as confirmed by immunostaining (PBS: n=4, Prop: n=3 hearts for P4; n=4 hearts/group for P8) (FIG. 12C) and total number of cardiomyocytes (quantified by counting fixation-digested hearts, PBS: n=7, Prop: n=6 hearts for P4; n=4 hearts/group for P8) after Propranolol administration (Prop, 10 μg/g body, 2 i.p. injections per day in newborn mice) (FIG. 12D). Quantification of cell cycle entry, as confirmed by immunostaining (n=5 hearts/group for P8) (FIG. 12E) and M-phase activity (n=4 hearts/group for P8) (FIG. 12F). Quantification of multinucleated cardiomyocytes (FIG. 12G) and total number of cardiomyocytes (quantified by counting fixation-digested hearts) after Alprenolol administration, as confirmed by immunostaining (Alp, 10 μg/g body, 2 i.p. injections per day in newborn mice, n=6 hearts/group) (FIG. 12H). Statistical significance was tested with Student's t-test (FIGS. 12A, 12E-12F), two-way ANOVA with Bonferroni's multiple comparisons (FIGS. 12B-12D, 12G-12H).

FIGS. 13A-13M: Propranolol-induced increase of the cardiomyocyte endowment in the neonatal period improves adult cardiac function and remodeling after MI. Mice received propranolol (Prop, 10 μg/g body, 2 i.p. injections per day, P1-12) or PBS. Quantification of cardiomyocytes in adult hearts at baseline (fixation-digested hearts, n=6 hearts/group for P42) (FIG. 13A). Ejection fraction of adult hearts at baseline (P60, n=11 hearts for PBS, n=6 hearts for Prop) (FIG. 13B). Diagram of experimental design. MI was induced by permanent ligation of the left anterior descending coronary artery between 6 weeks (P44) and 2 months after birth (P60). MRI was performed in the acute phase (1-3 dpi, days post injury) and recovery phase (10-12 dpi) (FIG. 13C). MRI (FIG. 13D) of late gadolinium enhancement (LGE) in both groups in acute and recovery phases. MI size is indicated and quantified (n=6 mice/group for acute phase; PBS: n=7, Prop: n=6 mice for recovery phase) (FIG. 13E). MRI images (FIG. 13F) and quantification of ejection fraction in the acute and recover phase in propranolol- or PBS-treated mice (PBS: n=4, Prop: n=5 mice for acute phase; PBS: n=7, Prop: n=6 mice for recovery phase) (FIG. 13G). MRI images (FIG. 13H) and analysis of stretched myocardial wall (PBS: n=5, Prop: n=4 mice for recovery phase) (FIG. 13I). Analysis of systolic myocardial thickening in the acute phase, as confirmed by MRI (FIG. 13J, PBS: n=4, Prop: n=5 mice) and the recovery phase (FIG. 13J, PBS: n=7, Prop: n=6 mice). The scar size quantified by AFOG staining (PBS: n=5, Prop: n=4 hearts for recovery phase) at 12 dpi (FIG. 13K). Number of cardiomyocytes (determined by stereology, n=5 hearts/group) (FIG. 13L) and heart weight to body weight ratio (PBS: n=5, Prop: n=6 hearts) (FIG. 13M). Statistical significance was tested with Student's t-test (FIGS. 13A-13B, 13I, 13K-13L), and one-way ANOVA with Bonferroni's multiple comparisons (FIGS. 13E-13G, 13J).

FIGS. 14A-14F: β-adrenergic signaling regulates cytokinesis in cardiomyocytes from patients with ToF/PS. Expression of cell cycle and cytokinesis-related genes in cycling cardiomyocytes from patients with ToF/PS and non-ToF/PS fetuses. Each symbol represents one cycling cardiomyocyte (Fetal: n=66 cardlomyocytes from 4 hearts, ToF/PS: n=14 cardiomyocytes from 3 hearts) (FIG. 14A). Ect2-positive cycling cardiomyocytes in hearts (Fetal: n=4 hearts, ToF/PS: n=3 hearts) (FIG. 14B). Quantification of cytokinesis failure in cultured human fetal cardiomyocytes, as confirmed by immunostaining (Ctrl: control; Fsk: Forskolin, Prop: Propranolol; Dobu: Dobutamine: 10 μM), measured by formation of binucleated daughter cells (n=isolations from 4 hearts) (FIG. 14C). Analysis of Ect2-positive midbodies (n=isolations from 2 hearts) (FIG. 14D). Analysis of cytokinesis in cultured myocardium (n=cultures from 3 patients with ToF/PS) treated with Fsk, Dobu, or Dobu+Prop. The interrupted red line indicates maximal cytokinesis failure induced by Fsk (positive control) (FIG. 14E). Proposed model connecting cytokinesis failure to endowment changes (FIG. 14F). Statistical significance was tested with Student's t-test (FIG. 14B) and one-way ANOVA with Bonferroni's multiple comparisons test (FIGS. 14C-14E).

FIGS. 15A-15F: Knockdown of Ect2 using siRNA reduces Ect2 mRNA and protein and induces cytokinesis failure and binucleation in cardiomyocytes. Two Ect2 siRNAs were tested in cultured fetal mouse cardiomyocytes and reduced Ect2 mRNA (FIG. 15A) and protein (FIG. 15C). The Western blot shows results from 3 independent experiments, separated by vertical lines (FIG. 15B). Ect2 siRNA #1 was selected for the experiments in FIG. 11G. Ect2 siRNA #2 knockdown of Ect2 increased binucleation of cultured fetal (E18) mouse cardiomyocytes (FIG. 15D). Ect2 siRNA #1 knockdown was performed in fetal cardiomyocytes isolated from mice expressing AG-Geminin and βMHC-YFP (to highlight sarcomeres) (FIGS. 15E and 15F). Live cell imaging (FIG. 15E) shows that Ect2 siRNA knockdown induces lack of cleavage furrow ingression and increase of cleavage furrow regression (siRNA Sc group, n=10 mitotic cardiomyocytes; siRNA Ect2 #1, n=14 mitotic cardiomyocytes). Statistical significance tested with one-way ANOVA with Bonferroni's multiple comparisons (FIGS. 15A, 15C), Student's t-test (FIG. 15D). Scale bar: 20 μm (FIG. 15E).

FIGS. 16A-16B: Altering β-AR signaling does not affect the heart weight. Propranolol-treatment (Prop, 10 μg/g body, 2 i.p. injections per day to newborn mice) P4: n=7 hearts with PBS, n=6 hearts with Prop; P8: n=4 hearts/group (FIG. 16A). Alprenolol-treatment (Alp, 10 μg/g body, 2 i.p. injections per day to newborn mice) n=6 hearts/group (FIG. 16B). Statistical significance was tested with two-way ANOVA with Bonferroni's multiple comparisons.

FIGS. 17A-17B: The total number of cardiomyocytes (FIG. 17A) and heart-body weight ratio (FIG. 17B) in neonatal mice administered metoprolol.

FIGS. 18A-18C: The total number of cardiomyocytes (FIG. 18A), heart-body weight ratio (FIG. 18B), and percent multinucleated (22) cardiomyocytes (FIG. 18C) in neonatal mice administered alprenolol.

### DETAILED DESCRIPTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise” or “comprised,” and variations thereof, are open ended and do not exclude the presence of other elements not identified. In contrast, the term “consisting of” and variations thereof is intended to be dosed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of a congenital heart disease or one or more defects relating to a congenital heart disease, means administration to a patient by any suitable dosage regimen, procedure and/or administration route of a composition, device, or structure with the object of achieving a desirable clinical/medical end-point, including but not limited to, for a congenital heart disease or one or more partial or complete repair of one or more defects relating to a congenital heart disease and/or amelioration of symptoms or sequelae thereof, an increase of cardiomyocyte endowment, e.g., by at least 5%, or improvement of a symptom of the congenital heart disease or defect, such as lowering to a normal range or toward a normal range of right ventricle pressure arising, e.g. from right ventricular hypertrophy associated with a congenital heart disease, such as tetralogy of Fallot. By “normal” in the context of a clinical value or a tissue structure or composition, it is meant a value that is found in or characterized in a normal patient or patient population. In this context, “normalization” refers to a value or characteristic within a normal range of values or characteristics for a patient or population, or approaching that normal value or characteristic from an abnormal value or characteristic, for example by application of a method or use described herein. An amount of any reagent or therapeutic agent, administered by any suitable route, effective to treat a patient is an amount capable of preventing, reducing, and/or eliminating any symptom op defect associated with a congenital heart defect. The effective amount of the beta blocker may range from 1 μg per dose to 10 g per dose, including any amount there between, such as, for example and without limitation, 1 ng, 1 μg, 1 mg, 10 mg, 100 mg, or 1 g per dose. The therapeutic agent may be administered by any effective route, and, for example, may be administered continuously, or at regular or irregular intervals, in amounts and intervals as dictated by any clinical parameter of a patient.

The compositions described herein can be administered by any effective route, such as parenteral, e.g., intravenous, intramuscular, subcutaneous, intradermal, etc., formulations of which are described below and in the below-referenced publications, as well as is broadly-known to those of ordinary skill in the art.

Active ingredients, such as small molecule drugs, oligomeric or polymeric compositions, polysaccharides, proteins, peptides, or nucleic acids or analogs thereof, may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms include: intravenous, intramuscular, or intraperitoneal solutions, oral tablets or liquids, topical ointments or creams and transdermal devices (e.g., patches). In one embodiment, the compound is a sterile solution comprising the active ingredient (drug, or compound), and a solvent, such as water, saline, lactated Ringers solution, or phosphate-buffered saline (PBS). Additional excipients, such as polyethylene glycol, emulsifiers, salts and buffers may be included in the solution.

Suitable dosage forms may include single-dose, or multiple-dose vials or other containers, such as medical syringe or intravenous (i.v.) bags containing a beta blocker. Numerous beta blocker formulations or dosage forms are commercially-available and may be compounded in a pharmacy for delivery according to the methods described herein.

Pharmaceutical formulations adapted for parenteral administration include aqueous and non-aqueous sterile injection solutions which may contain, for example and without limitation, anti-oxidants, buffers, bacteriostats, lipids, liposomes, emulsifiers, also suspending agents and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions, such as parenteral or inhaled compositions, in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

A “beta blocker” (β-blocker) is a beta-adrenergic blocking agent. They bind to G-protein coupled receptors, known as beta-adrenergic receptors (beta-adrenoceptors or β-AR), and block the binding of norepinephrine, epinephrine, and other substances to those receptors, thereby inhibiting their normal sympathetic effects. Three types of beta-adrenoceptors have been distinguished, designated beta-1 (β₁), beta-2 (β2), and beta-3 (β₃) adrenoceptors. Generally, activation of β₁-ARs and β₂-ARs increases heart contractile force and heart rate and vascular and non-vascular smooth muscle relaxation. Beta blockers may be non-selective (first generation), blocking both β₁-ARs and β₂-ARs, or selective, which commonly block β₁-ARs, but can block β₂-ARs at higher doses.

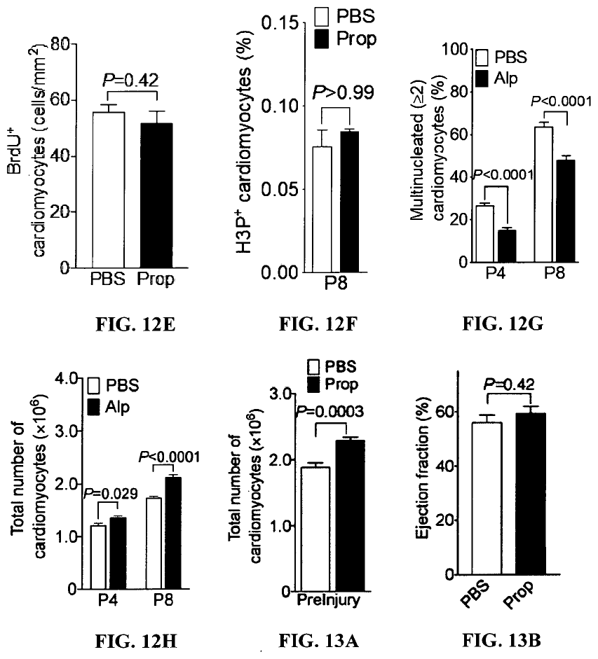

Non-selective beta blockers, β₁-selective beta-blocker agents, and/or β₂-selective beta-blocker agents may be used in methods described herein. As such, corresponding uses for non-selective beta blockers, β₁-selective beta-blocker agents, and/or β₂-selective beta-blocker agents are provided herein. Non-specific beta-blockers, such as propranolol (1-naphthalen-1-yloxy-3-(propan-2-ylamino)propan-2-ol) or alprenolol (1-(propan-2-ylamino)-3-(2-prop-2-enylphenoxy)propan-2-ol), which block both β₁-ARs and β₂-ARs, are seen to be effective (FIGS. 12C-12H, 13A, 13B, 13E, 13G, 13I-13M, 14C, 14D, 14E, 16A, 16B, 18A-18C. The beta blocker can be a non-specific beta blocker or a β-selective beta-blocker.

Non-limiting examples of non-specific beta blockers include: propranolol, bucindolol (e.g., 2-[2-hydroxy-3-[[1-(1H-indol-3-yl)-2-methylpropan-2-yl]amino]propoxy]benzonitrile), carteolol (5-[3-(tert-butylamino)-2-hydroxypropoxy]-3,4-dihydro-1H-quinolin-2-one), carvediol (e.g., 1-(9H-Carbazol-4-yloxy)-3-[[2-(2-methoxy-d3-phenoxy)ethyl]amino]-2-propanol), labetalol (e.g., 2-hydroxy-5-[1-hydroxy-2-(4-phenylbutan-2-ylamino)ethyl]benzamide), nadolol (e.g., (2R,3S)-5-[3-(tert-butylamino)-2-hydroxypropoxy]-1,2,3,4-tetrahydronaphthalene-2,3-diol), oxprenolol (e.g., 1-(propan-2-ylamino)-3-(2-prop-2-enoxyphenoxy)propan-2-ol), penbutolol (e.g., (2S)-1-(tert-butylamino)-3-(2-cyclopentylphenoxy)propan-2-ol), pindolol (e.g., 1-(1H-indol-4-y4oxy)-3-(propan-2-ylamino)propan-2-ol), sotalol (e.g., N-[4-[1-hydroxy-2-(propan-2-ylamino)ethyl]phenyl]methanesulfonamide), and timolol (e.g., (2S)-1-(tert-butylamino)-3-[(4-morpholin-4-yl-1,2,5-thiadiazol-3-yl)oxy]propan-2-ol), including any pharmaceutically-acceptable salt thereof, though some may be preferred that have no additional activity, such as α1-blocking activity or intrinsic sympathomimetic activity.

β₁-selective beta-blocker agents include, for example and without limitation, butaxamine (e.g., 2-(tert-butylamino)-1-(2,5-dimethoxyphenyl)propan-1-ol) and ICl-118,551 e.g., (3-(isopropylamino)-1-[(7-methyl-4-indanyl)oxy]butan-2-ol), including any pharmaceutically-acceptable salt thereof. β₁-selective beta-blocker agents include, for example and without limitation, atenolol (e.g., 2-[4-[2-hydroxy-3-(propan-2-ylamino)propoxy]phenyl]acetamide), metoprolol (e.g., 1-[4-(2-methoxyethyl)phenoxy]-3-(propan-2-ylamino)propan-2-ol), nebivolol (e.g., 1-(6-fluoro-3,4-dihydro-2H-chromen-2-yl)-2-[[2-(6-fluoro-3,4-dihydro-2H-chromen-2-yl)-2-hydroxyethyl]amino]ethanol) and bisoprolol (e.g., 1-(propan-2-ylamino)-3-[4-(2-propan-2-yloxyethoxymethyl)phenoxy]propan-2-ol), including any pharmaceutically-acceptable salt thereof.

β₁-selective beta-blocker agents include, for example and without limitation, atenolol, betaxolol, bisoprolol, esmolol, acebutolol, metoprolol, and nebivolol.

Cardiomyocyte endowment refers to the total number of cardiomyocytes in a mature subject. The methods and uses described herein are applicable to a pediatric patient population that is capable of undergoing cardiomyocyte expansion and is experiencing a heart disease that includes suppressed cytokinesis. At a point during development (which is after birth in humans, but before they are adults), cardiomyocytes do not proliferate anymore. In other words: sufficient numbers of mononuclear cardiomyocytes, which give rise to proliferation, are no longer present to produce a normal cardiomyocyte endowment, even when stimulated with a beta blocker, and as such, the treatments and uses described herein are subject to the availability of mononuclear cardiomyocytes. Sufficient numbers of mononuclear cardiomyocytes to produce a normal cardiomyocyte endowment are present at birth. In humans without heart disease, cardiomyocytes can proliferate for up to 10 years after birth. That said, in patients with heart disease having suppressed cytokinesis, addressing the suppressed cytokinesis at a time when the potential for cardiomyocyte expansion is maximized, and, in many instances, prior to development significant acute symptoms of the heart disease requiring significant therapeutic intervention or palliative care, such as cyanosis. As such, the treatments and uses described herein may be primarily applicable to infants (patients) less than six months post term, including neonates (preterm infants and term newborn infants, e.g. up to one month or 27 days past term). The definition of “pediatric” patients may vary to some extent, depending on, e.g., developmental biology and pharmacology. “Pediatric” may be defined as a patient who is 21 years old or younger, 18 years old or younger, 17 years old or younger, 16 years old or younger, or 15 years old or younger, and may be defined by regulation or an appropriate regulatory agency (e.g., in the United States, as determined pursuant to 21 U.S.C. 355a and/or by the U.S. Food and Drug Administration). The following is one possible categorization, including pediatric subclasses, recognizing that there is considerable overlap in developmental issues across the age categories.
Preterm infants;
Term newborn infants (0 to 27 days);
Infants and toddlers (28 days to 23 months);
Children (2 to 11 years); and
Adolescents (12 to 16-18 years.

“Term” refers to the normal gestational period for a human, which can be measured by any effective method, such as from last menstrual period, or any other acceptable method of determining gestational period. Term often ranges from 37 to 42 weeks, and may be 40 weeks or 280 days. “Past term” is the post-term time elapsed after the term date of an infant, and, as an example, using 40-months as “term”, where the infant was born at 40 months, a date that is one month after birth is one month past term. Likewise, an infant born pre-term at 35 months, again designating 40-months as “term”, is one month past term six months after birth.

The process of building the cardiomyocyte endowment predominantly occurs pre-term, in term newborn infants, and into infancy, typically until six months past term, but can continue, though at a much lesser extent, through toddlerhood and childhood, and can even extend into adolescence. As such, while the methods described herein may be most relevant to pre-term infants, term newborn infants, and infants less than six months past term, efficacy and treatment may include treatment of children, adolescents, and even into adulthood. Further, treatment may be initiated any time during development or afterward, so long as cardiomyocyte endowment can be increased by administration of beta blockers as described herein. Irrespective of the time of initial administration of the beta blockers, treatment may continue until a normal cardiomyocyte endowment is achieved, or a sufficient therapeutic effect is achieved, and may be initiated at any relevant pediatric stage, and can continue through later pediatric stages and into adulthood. Treatment may be terminated at any time once cardiac sufficiency is attained (e.g., normal heart structure or function) or clinically acceptable or clinically sufficient improvement of heart structure or function is attained, e.g. as determined by any suitable method, such as by ultrasound (e.g. echocardiogram), electrocardiogram (ECG or EKG), cardiac MRI, by heart biopsy indicating acceptable, normal, or near normal percentages of binucleated or multinucleated cardiomyocytes, or any other useful metric or measure of patient cardiac sufficiency or health. In patients with acutely life-threatening defects, treatment may start after surgery to correct a heart defect in order to expand the patient's cardiomyocyte endowment, thereby lowering the risk of future myocardial infarct. Treatment may be conducted prior to and after surgery to correct a heart defect to expand the patient's cardiomyocyte endowment, thereby lowering the risk of complications associated with future myocardial infarction, such as heart failure.

Treatment with beta blockers according to the methods and uses described herein may follow identification of cytokinesis failure in a patient less than six months past term. A heart biopsy may be obtained, e.g., by catheterization, in a patient less than six months past term, having a CHD or heart defect. Analysis of the biopsy may indicate abnormally high numbers of binucleated cardiomyocytes (above an expected normal range). While the percentage of binucleated cardiomyocytes may be normal (e.g., approximately 20% at birth), the percentage of binucleated cardiomyocytes may rapidly increase after birth and before six months past term to 30%, 40%, or 50%, or any increment therebetween after birth. Further, patients exhibiting cytokinesis failure less than six months past-term may have multinucleated (>2 nuclei) cardiomyocytes, which is a strong indicator of cytokinesis failure, and which would benefit from beta-blocker treatment as indicated herein.

Typically CHD is diagnosed very early in the life of a patient, before birth, as a preterm infant, a term newborn infant, or as an infant or toddler. Once CHD is diagnosed, the patient, e.g., the pediatric patient, may be administered the beta blocker, optionally with one or more additional therapeutic agents, and monitored for improvement of heart structure and/or function accordingly. Treatment is not acute, as in the administration of a single bolus or repeated administration over a short period, such as over less than one week, or less than one day. Treatment is continuous and is continued for a sufficient time to achieve an improvement of heart structure and/or function, for example for at least two weeks, at least 1 month, at least 2 months, at least 3 months, at least 4 months, at least 5 months, at least 6 months, at least 7 months, at least 8 months, at least 9 months, at least 10 months, at least 11 months, or at least one year. Suitable dosage ranges may be the same as for current use of beta blockers, and can vary depending on the therapeutic window or therapeutic index for any chosen drug, e.g. as currently determined for known beta blockers, or as determined in pediatric patient. Therapeutic window or therapeutic index refer to the range of doses at which a medication is effective without unacceptable adverse events. For example, propranolol may be administered in a range of from 0.01 mg/kg/day to 20 mg/kg/day in pediatric patients. Dosages may be titrated depending on individual patient tolerance.

In one example, propranolol may be administered at 1 mg/kg/day PO given in divided doses every 6 hours. After 1 week, dosage may be titrated by 1 mg/kg/day every 24 hours as necessary to a maximum of 5 mg/kg/day.

Cardiomyocyte endowment refers to the overall number of cardiomyocytes in the heart of a patient. Normal cardiomyocyte endowment in adult humans is approximately four billion (4×10⁹), and in mice is approximately two to four million (4×10⁶). A “low cardiomyocyte endowment” refers to a patient comprising a cardiomyocyte endowment reduced statistically significantly in a patient so as to result in a heart defect, such as a septal defect, or any defect, for example as indicated below. Cardiomyocyte endowment in living patients may be measured or estimated in any art-acceptable manner, for example by use of ultrasound methods, such as echocardiography that may be combined with suitable computer-implemented methods to estimate volume or mass of any heart structure or the heart in its entirety. Cardiomyocyte endowment also may be estimated by the presence of and severity of any heart defect, such as septal defects, patent foramen ovale, valve defects,

A congenital heart disease is a condition in which one or more structural heart defects is present in the heart of a patient. Congenital heart diseases may coincide with a low cardiomyocyte endowment, which typically is reduced by 1% to 20% or 25%, or more, until a percentage that is so significant that one or more structural defects result that are so severe that a fetus or infant would not survive. Congenital heart diseases or defects involving low cardiomyocyte endowment include, without limitation: tetralogy of Fallot; tetralogy of Fallot with pulmonary valve stenosis; aortic stenosis; coarctation of the aorta; Ebstein's anomaly; patent ductus arteriosus; pulmonary valve stenosis; s septal defect, such as an atrial septal defect or an ventricular septal defect; a single ventricle defect, such as hypoplastic left heart syndrome or tricuspid atresia; total or partial anomalous pulmonary venous connection (TAPVC); transposition of the great arteries; or truncus arteriosus, and as, such the methods and uses described herein may be used to treat a patient having such defects, with or without corrective surgery to increase cardiomyocyte endowment in the patient and, to correct the defect or one or more defects associated with a CHD. Defects associated with tetralogy of Fallot include: pulmonary valve stenosis, a ventricular septal defect, an overriding aorta, and right ventricular hypertrophy, and may be considered as an anterior malalignment of the infundibular septum with the muscular septum. Additional defects may be present in tetralogy of Fallot, such as a hypoplastic or absent conal septum (e.g. latin or Mexican tetralogy of Fallot), stenosis of the left pulmonary artery, a bicuspid pulmonary valve, a right-sided aortic arch, coronary artery anomalies, a patent foramen ovale or atrial septal defect, an atrioventricular septal defect, a partial or complete pulmonary vein return anomaly, and/or pulmonary atresa. Defects associated with trilogy of Fallot include: pulmonary valve stenosis, right ventricular hypertrophy, and an atrial septal defect.

By low cardiomyocyte endowment, it is meant an overall number of cardiomyocytes (heart muscle cells) than are present in a typical normal patient or population of normal patients. Patients with low cardiomyocyte endowment approaching normal endowment, such as from 1% to 5% lower than normal endowment, may not show significant impairment or structural defects, but may be treated to expand the cardiomyocyte endowment. Even patients with moderately or severely low cardiomyocyte endowment, e.g., ranging from 5% to 20% lower endowment as compared to normal, and exhibiting substantial heart defects, may not exhibit overt hypoxia, cyanosis, or other symptoms of the defect, until after infancy. Classically, beta blockers have been administered to pediatric patients suffering from tetralogy of Fallot as a palliative treatment for cyanosis and other symptoms that typically occur after infancy, e.g., after six months of age.

Hypoxemia or hypoxemic refers to below normal oxygen partial pressure values in the blood (PaO2 or PO2), with values of less than 60 mmHg, being considered as moderately or severe hypoxemic. Hypoxia or hypoxic can be a result of hypoxemia and refers to low tissue oxygenation. Hypoxia may be measured with a pulse oximeter, with SpO2 values of less than 90% at sea level being typically considered to be hypoxic. Cyanosis or cyanotic refer to tissue bluing or purpling as a result of hypoxia and is often first seen in peripheral tissue (peripheral cyanosis) such as fingers, nail beds, lips, and tongue, but can progress to systemic or central cyanosis. Cyanosis may be seen with SpO2 levels of 85% or less.

In aspects or embodiments, the methods and uses described herein may require not only administration of the beta blocker to the patient, but testing the patient to ascertain the presence of cytokinesis failure, and, once treatment is performed, to ascertain the extent of improvement of one or more aspects of the congenital heart disease, such as correction of an associated defect, normalization of the number of cardiomyocytes in the patient's heard, normalization of the percentage of binucleated cardiomyocytes or multinucleated cardiomyocytes in heart tissue of the patient, or normalization of a physiological parameter without any palliative treatment concurrent with measurement of the parameter, such as administration of beta blockers or presence of therapeutic amounts of a beta blocker in the patient's system at the time of testing. Testing may be performed in any manner. For example and without limitation, repair of structural defects may be monitored by imaging, such as by ultrasound, and commonly be echocardiography. Correction of blood flow patterns may likewise be monitored by echocardiography. The percentage of binucleated or multinucleated cardiomyocytes may be determined by taking a biopsy of heart tissue, e.g., by catheterization, and determining by any suitable pathological technique the overall cardiac endowment or percentage of binucleated or multinucleated cardiomyocytes in the biopsy. Physiological values, such as tissue oxygen or blood pressure in the heart, may be monitored (determined or ascertained at one or more time points during treatment), e.g. at regular intervals such as weekly, bi-weekly, monthly, bi-monthly, quarterly, or semiannually, or at any suitable interval. Right ventricle systolic pressure (RVSP) may be monitored directly by catheterization, for example at the same time a biopsy is obtained, or indirectly estimated by echocardiography. Blood oxygen (e.g. SpO2) may be measured by pulse oximeter. Mass of the heart or any structure of the heart, such as ventricular muscle mass, may be determined by imaging, computer, or microscopy analysis. Mass of the heart or a structure thereof, such as ventricular muscle mass, may be used to estimate cardiomyocyte endowment or the degree of right ventricle hypertrophy in a patient. Multiple different assays may be performed at one or more time points during the course of treatment. Suitable end-points for repair of any given defect, measure of cardiomyocyte endowment, or for any physiological parameter, such as RVSP or SpO2, may be ascertained in any manner, but normalization of affected structures, cardiomyocyte endowment, or the physiological parameter would be an overall goal of the treatments and uses described herein.

The methods and uses described herein may be used for treatment of patients, e.g., pre-term infants, term newborn infants, or infants less than 6 months past term, for elevated RVSP, which may be measured, and typically is measured, by catheter, or estimated by echocardiography. RVSP can be measured before treatment with beta blockers, and administration of beta blockers can be continued until RVSP levels normalize (reach an age-appropriate normal value or are lowered acceptably towards a normal value). Normal RVSP for pre-term and infants less than 6 months old may range from 15 mmHg to 30 mmHg. RVSP may be measured without a beta blocker present in the patient's system at therapeutic levels to assure the normalized RVSP continues without the presence of the beta blocker in the patient's system. However, measurement of RVSP is not necessarily required for the effectiveness of beta blocker, e.g. propranolol, administration, nor is normalization of the RVSP.

The method may combine propranolol administration with administration of an agent that increases cardiomyocyte cell cycle entry and progression, for example neuregulin or fibroblast growth factor (FGF), or any other protein or chemical that stimulates cardiomyocyte cell cycle activity to cytokinesis.

A method of treating a patient having a heart defect, e.g., a congenital heart defect or disease, and a low or reduced cardiomyocyte endowment resulting from cytokinesis failure, according to any aspect, embodiment, or example described herein, is provided that combines beta-blocker, e.g., propranolol, administration with administration of one or more additional second therapeutic agents. The one or more additional second therapeutic agent may be a growth factor, which optionally may be prepared using recombinant techniques. Non-limiting examples of growth factors include basic fibroblast growth factor (bFGF), acidic fibroblast growth factor (aFGF), vascular endothelial growth factor (VEGF), Human Vascular Endothelial Growth Factor-165 (hVEGF165), Vascular endothelial growth factor A (VEGF-A), Vascular endothelial growth factor B (VEGF-B), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Califomia; ProSpec-Tany TechnoGene Ltd., Rehovot, Israel; and Cell Sciences®, Canton, Mass.

The one or more additional second therapeutic agent may be an antimicrobial agent, such as, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, dindamycin, lincomycin, pentamidine, atovaquone, paromomycin, diclazaril, acyclovir, trifluorouridine, foscamet, penicillin, gentamicin, gancidovir, iatroconazole, miconazole, Zn-pyrithione, and silver salts such as chloride, bromide, iodide, and periodate.

The one or more additional second therapeutic agent may be an anti-inflammatory agent, such as, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin; nitro-fatty acids, such as nitro-oleic acid or nitro-conjugated linoleic acid. Other drugs that may promote wound healing and/or tissue regeneration may also be included as the one or more additional second therapeutic agents.

A method of treating a patient having a heart defect, e.g., a congenital heart defect or disease, and a low or reduced cardiomyocyte endowment resulting from cytokinesis failure, according to any aspect, embodiment, or example described herein, is provided that combines beta-blocker, e.g., propranolol, administration with administration of an agent that increases cardiomyocyte cell cycle entry and progression, or any other protein or chemical that stimulates cardiomyocyte cell cycle activity to cytokinesis, for example and without limitation, periostin, neuregulin, or fibroblast growth factor (FGF). The method can comprises administering to the patient a beta blocker as described herein, in combination with a second therapeutic agent. The second therapeutic agent may be compound or composition such as a growth factor or mitogen, for stimulating cardiomyocyte cell cycle entry and/or progression. The second therapeutic agent can be periostin, neuregulin, or a fibroblast growth factor. A growth factor is a substance, such as a peptide or protein that stimulates cell growth, while a mitogen is generally a peptide or small protein that induces a cell to begin mitosis. Growth factors are broadly-characterized and are well-known, and can stimulate cardiomyocyte cell cycle entry and/or progression, and cardiomyocyte proliferation. Non-limiting examples of useful growth factors include, without limitation, fibroblast growth factors and neuregulins. An example of a useful fibroblast growth factor is Fibroblast Growth Factor 2 (FGF-2), as is broadly-known.

Neuregulins (NRGs) are members of the epidermal growth factor (EGF) family of proteins. In one example, the neuregulin is a neuregulin 1 (NRG-1) protein, which is a cardioactive growth factor released from endothelial cells that is necessary for cardiac development, structural maintenance, and functional integrity of the heart. NRGs are described, for example, in United States Patent Application Publication No. 20160095903, which is incorporated herein by reference in its entirety for its technical disclosure.

Neuregulin or NRG refers to proteins or peptides that can bind and activate ErbB2, ErbB3, ErbB4 or combinations thereof, including but not limited to all neuregulin isoforms, neuregulin EGF domain alone, polypeptides comprising neuregulin EGF-like domain, neuregulin mutants or derivatives, and any kind of neuregulin-like gene products that also activate the above receptors as described below and, for example, in US 20160095903 A1. Neuregulin may bind to and activate ErbB2/ErbB4 or ErbB2/ErbB3 heterodimers. Neuregulin can activate the above ErbB receptors and modulate their biological reactions, e.g., stimulate breast cancer cell differentiation and milk protein secretion; induce the differentiation of neural crest cell into Schwann cell; stimulate acetylcholine receptor synthesis in skeletal muscle cell; and/or improve cardiocyte differentiation, survival and DNA synthesis. Assays for measuring the receptor binding activity are known in the art. For example, cells transfected with ErbB-2 and ErbB-4 receptor can be used. After receptor expressing cells are incubated with excess amount of radiolabeled neuregulin, the cells are pelleted and the solution containing unbound radiolabeled neuregulin is removed before unlabeled neuregulin solution is added to compete with radiolabeled neuregulin. EC₅₀ is measured by methods known in the art. EC₅₀ is the concentration of ligands which can compete 50% of bound radiolabeled ligands off the receptor complex. The higher the EC₅₀ value is, the lower the receptor binding affinity is.

“Neuregulin” includes any neuregulin and isoforms thereof known in the art, including but not limited to all isoforms of neuregulin-1 (“NRG-1”), neuregulin-1 (“NRG-2”), neuregulin-1 (“NRG-3”) and neuregulin-4 (“NRG-4”). NRG-1 is described, for example, in U.S. Pat. Nos. 5,530,109, 5,716,930, and 7,037,888. NRG-2 is described, for example, in International Pat Pub. No. WO 97/09425). NRG-3 is described, for example, in Hijazi et al., 1998, Int. J. Oncol. 13:1061-1067. NRG-4 is described, for example, in Harari et al., 1999 Oncogene. 18:2681-89. “Neuregulin” may comprise the EGF-like domain encoded by NRG-2. Neuregulin may comprise the EGF-like domain encoded by NRG-3. Neuregulin may comprise the EGF-like domain encoded by NRG-4. Neuregulin may comprise the amino acid sequence of Ala Glu Lys Glu Lys Thr Phe Cys Val Asn Gly Gly Glu Cys Phe Met Val Lys Asp Leu Ser Asn Pro (SEQ ID NO: 1), as described in U.S. Pat. No. 5,834,229.

A neuregulin may be a fragment or variant of neuregulin-1 (NRG-1) or neuregulin-1β, e.g., as described in U.S. Pat. No. 9,340,597 incorporated herein by reference for its technical disclosure. The neuregulin may be a peptide as described in U.S. Pat. No. 9,340,597, incorporated herein by reference for its technical disclosure, such as the following:

Neuregulin-1 (NRG-1) includes without limitation: NRG61 (e.g., NEUCARDIN™), having the amino acid sequence:

(SEQ ID NO: 2)
Ser His Leu Val Lys Cys Ala Glu Lys Glu Lys Thr
Phe Cys Val Asn Gly Gly Glu Cys Phe Met Val Lys
Asp Leu Ser Asn Pro Ser Arg Tyr Leu Cys Lys Cys
Pro Asn Glu Phe Thr Gly Asp Arg Cys Gln Asn Tyr
Val Met Ala Ser Phe Tyr Lys Ala Glu Glu Leu Tyr
Gln,

which corresponds to amino acids 177-237 of human NRG-1.
Neuregulin peptide EGF53, having the amino acid sequence:

(SEQ ID NO: 3)
Ser His Leu Val Lys Cys Ala Glu Lys Glu Lys Thr
Phe Cys Val Asn Gly Gly Glu Cys Phe Met Val Lys
Asp Leu Ser Asn Pro Ser Arg Tyr Leu Cys Lys Cys
Pro Asn Glu Phe Thr Gly Asp Arg Cys Gln Asn Tyr
Val Met Ala Ser Phe.

Neuregulin peptide NRG55, having the amino acid sequence:

(SEQ ID NO: 4)
Ser His Leu Val Lys Cys Ala Glu Lys Glu Lys Thr
Phe Cys Val Ash Gly Gly Glu Cys Phe Met Val Lys
Asp Leu Ser Ash Pro Ser Arg Tyr Leu Cys Lys Cys
Pro Asn Glu Phe Thr Gly Asp Arg Cys Gln Asn Tyr
Val Met Ala Ser Phe Tyr Lys,

Neuregulin peptide NRG57, having the amino acid sequence:

(SEQ ID NO: 5)
Ser His Leu Val Lys Cys Ala Glu Lys Glu Lys Thr
Phe Cys Val Asn Gly Gly Glu Cys Phe Met Val Lys
Asp Leu Ser Ash Pro Ser Arg Tyr Leu Cys Lys Cys
Pro Asn Glu Phe Thr Gly Asp Arg Cys Gln Asn Tyr
Val Met Ala Ser Phe Tyr Lys Ala Glu,

Neuregulin peptide NRG59, having the amino acid sequence:

(SEQ ID NO: 6)
Ser His Leu Val Lys Cys Ala Glu Lys Glu Lys Thr
Phe Cys Val Asn Gly Gly Glu Cys Phe Met Val Lys
Asp Leu Ser Asn Pro Ser Arg Tyr Leu Cys Lys Cys
Pro Asn Glu Phe Thr Gly Asp Arg Cys Gln Asn Tyr
Val Met Ala Ser Phe Tyr Lys Ala Glu Glu Leu.

Additional information regarding neuregulin, e.g., NRG-1 may be found in United States Patent Application Publication Nos. 20140364366, 20170189489, and 20200031892 and U.S. Pat. No. 9,434,777, each of which is incorporated herein by reference in its entirety for its technical disclosure of neuregulins.

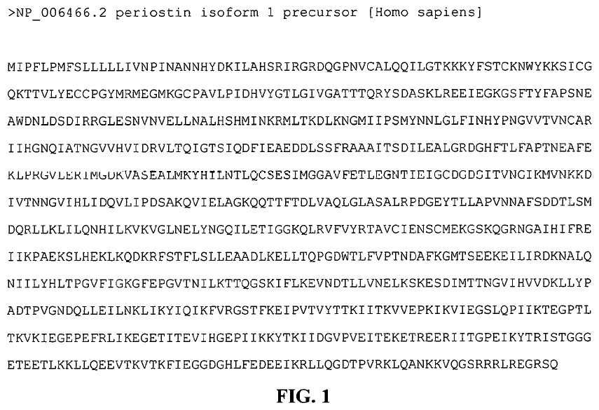

Periostin (e.g., POSTN) is a component of the extracellular matrix, and periostin and fragments thereof promote cardiomyocyte proliferation and myocardial regeneration, see, e.g. U.S. Pat. No. 8,936,806, which is incorporated herein by reference in its entirety for its technical disclosure and which describes periostin and with the examples describing the cardiomyocyte mitogenic activity of periostin, either as full-length periostin or in the form biologically active fragments thereof. Various isoforms are characterized, e.g., isoforms 1-8 as indicated in Gene ID: 10631 (National Center for Biotechnology Information, U.S. National Library of Medicine), with, as an example, isoform 1, as disclosed in NCBI Reference Sequence: NP_006466.2, as shown in FIG. 1 (SEQ ID NO: 7; see, also, SEQ ID NO: 1 of U.S. Pat. No. 8,936,806). Extracellular periostin can induce cell cycle re-entry of differentiated mammalian cardiomyocytes. Periostin stimulates mononuclear cardiomyocytes, present in the mammalian heart, to undergo the full mitotic cell cycle. Periostin-induced cardiomyocyte proliferation results from activation the ERK1/2 and Akt signaling pathways. “Periostin” can refer to full-length periostin, e.g. as shown in FIG. 1 and biologically-active periostin fragments, e.g., a portion or part of periostin comprising a fas1 domain, are described in U.S. Pat. No. 8,936,806, and biologically-active variants thereof, including naturally occurring alleles and homologs thereof.

Other therapeutic agents may be administered concurrently with the beta blocker, and optionally the growth factor or mitogen.

### EXAMPLES

Unlike adults in whom there is little proliferation of cardiomyocytes, heart muscle in infants and children without heart disease grows by proliferation and differentiation of cardiomyocytes. Cardiomyocyte proliferation after birth contributes to growth of the heart until it reaches adult size. No new cardiomyocytes are generated after the final number of cardiomyocytes, the endowment, is established. This number is approximately 4 billion in humans and 2 million in mice. Reduction in the endowment can result in heart failure with immediate onset or delayed onset. Heart failure in the setting of decreased endowment may also be precipitated by heart disease. The most graphic example is acute myocardial infarction, which can wipe out up to 1 billion cardiomyocytes, i.e., 25% of the cardiomyocyte endowment in an adult, and is associated with 50% mortality.

As shown in the Examples, below, infants with CHD have reduced proliferation of heart muscle cells (cardiomyocytes). Our results show that patients with CHD have a 20-30% lower endowment, as a result of insufficient division of cardiomyocytes.

We have identified a previously unrecognized molecular connection between the cellular mechanisms of endowment regulation, novel and unique molecular pathway implicated in CHD that can be targeted to design new therapeutics. Patients with CHD exhibit a higher percentage of binucleated heart muscle cells (cardiomyocytes), which cannot divide and, as such, do not contribute to proliferative heart growth and regeneration. In CHD, mononucleated cardiomyocytes are converted to binucleated cardiomyocytes and stop proliferating prematurely, leading to a smaller cardiomyocyte endowment. Our studies have indicated repression of the cytokinesis gene Ect2 as the central molecular change in binucleating cardiomyocytes. Inactivating Ect2 in cardiomyocytes during development results in a 3-fold increase in binucleation, a 50% decrease in cardiomyocyte endowment, and 100% lethality at postnatal day 2. Our data demonstrates that the Hippo tumor suppressor pathway represses Ect2 gene transcription, and that this occurs downstream of β-adrenergic receptors (β-AR). β-AR represent formidable and clinically validated pharmacologic targets, which provides a therapeutic opportunity for patients with CHD. Our results in mice have indicated that administration of β-blockers delays the formation of binucleated cardiomyocytes, increases cardiomyocyte endowment and promotes myocardial regeneration in mice.

Beta blockers are a class of drugs that are used to manage abnormal heart rhythms, tachyarrhythmia, hyperkinetic heart syndrome, coronary artery disease (CAD) and prophylaxis of myocardial infarction primarily through the blockade of cardiac β-receptors. Long term use of beta-blockers also helps to manage chronic heart failure and high blood pressure. A number of different β-adrenoceptor blockers, such as propranolol, atenolol, metoprolol, carvedilol, and bisoprolol, are approved for treatment of human cardiovascular disease.

### Example 1

We studied these mechanisms in Tetralogy of Fallot with pulmonary stenosis (ToF/PS), a common form of CHD that has relatively uniform structural defects (anterior deviation of the infundibulum, pulmonary stenosis, ventricular septal defect, and right ventricular hypertrophy). Despite extensive research to understand the genetic causes of ToF/PS, little is known at the molecular level. Although infants and children with ToF/PS rarely have heart failure, the disease leads to heart failure in adults, causing morbidity and mortality. This is currently explained with the sequelae of cardiac surgery. However, we have considered that cardiomyocyte changes may also occur in ToF/PS patients prior to surgery. We characterized changes in cardiomyocyte proliferation and differentiation in patients with ToF/PS by examining the prevalence and molecular mechanisms of binucleation in cardiomyocytes. Binucleation has previously been linked to the decrease of cardiomyocyte proliferative capacity; when cardiomyocytes stop proliferating in mice and rats in the first week after birth, they undergo incomplete cell cycles, leading to binucleated cardiomyocytes. Multiple studies have suggested that cardiomyocytes become binucleated by incomplete cytokinesis. The molecular alterations of the cytokinesis machinery in cardiomyocyte binucleation are largely unknown, as are the regulatory mechanisms.

By examining mouse cardiomyocytes in culture and in vivo, we sought to determine the cause of cytokinesis failure and its relationship to cardiomyocyte proliferation. Our present study demonstrates a function of β-AR signaling in regulating cardiomyocyte cytokinesis in vivo. Using formation of binucleated cardiomyocytes as read-out for the definitive endpoint of cell division, we discovered an extensive decrease in cardiomyocyte division in ToF/PS, causing a lack of endowment growth. We identified the mechanisms of formation of binucleated cardiomyocytes, establishing a new connection between β-AR signaling and regulation of cardiomyocyte cytokinesis.

### Materials and Methods

Study Design: The goal of this project was to determine if and how formation of binucleated cardiomyocytes is altered in ToF/PS. The study design, including the number of animals and the numbers of cells counted, was predefined by the investigators. The genotypes of all mice were recorded throughout the entire period of the project. For studies involving human tissue, the number of tissue samples was determined according to the availability of the samples. The investigators were blinded for the quantification of samples. Cardiomyocytes from patients with ToF/PS were collected as part of standard care during surgical repair. Human fetal myocardial samples were collected from abortions at 18 to 23 weeks gestation.

Genetically Modified Mice: Transgenic mAG-hGem mice were generated by Sakaue-Sawano et al. (Visualizing spatiotemporal dynamics of multicellular cell-cycle progression. Cell 132, 487-498 (2008)). mAG-hGem consists of a green fluorescent protein, mAG 339 (monomeric Azami Green), fused to the ubiquitination domain of truncated human Geminin at the N-terminus. To knockout the Ect2^{flox} gene before birth, Ect2^{flox} mice were crossed with αMHC-Cre (Agah et al. Targeted expression of Cre recombinase provokes cardiac-restricted, site-specific rearrangement in adult ventricular muscle in vivo. J Clin Invest 100, 169-179 (1997)) through breeding αMHC-Cre^{+/-};Ect2^{flox/+} mice with Ect2^{flox/flox} mice. To knockout the Ect2^{flox} gene after birth, we bred MHCMerCreMer^{+/-};Ect2^{flox/+} mice and administrated tamoxifen (30 micrograms per gram (μg/g) body weight) through intraperitoneal (i.p.) injection once daily from P0 to P2. The inactivation of Ect2^{flox} gene was confirmed by PCR analysis of genomic DNA, as previously described (Cook et al. The ect2 rho Guanine nucleotide exchange factor is essential for early mouse development and normal cell cytokinesis and migration. Genes Cancer 2, 932-942 (2011)). The mouse strain with β₁- and β₂-adrenergic receptor double knockout (DKO) was obtained from JAXR MICE and crossbred with wild type C57 mouse and inbred for at least 5 generations to generate DKO and appropriate control mice. Pregnant ICR CD1 mice (E16-E18) were purchased from Charles River.

Mouse Pharmacological Treatment: Forskolin (Fsk, 1 μg/g, 1 i.p. injection/day), propranolol (10 μg/g, 2 i.p. injections/day), or alprenolol (40 μg/g, 2 i.p. injections/day) were administered from P1 to P4 or P8 to P12. An equal volume of phosphate buffered saline (PBS) was injected in the control groups.

Mouse myocardial injury model: ICR mice were treated with propranolol (10 μg/g, 2 i.p. injections/day) from P1 to P12. Then, cardiac injury was induced to male mice via performing left anterior descending artery (LAD) ligation at the age of P40 to P60. Briefly, the mice were anesthetized with 4% isoflurane, intubated, and ventilated with a stroke volume of 225 microliters (μl) at 145 breaths/minute. The hearts were exposed by performing thoracotomy and the LAD coronary artery was tied with 8-0 nylon suture. After the chest was dosed with 6-0 suture, bupivacaine (8 μg/g) was locally administered through subcutaneous injection. The mice were kept intubated without isoflurane at room temperature until waking up from the anesthesia. Bupivacaine (8 μg/g) was administered by subcutaneous injection in the following 3 days (1 day post injury (dpi) to 3 dpi). The heart function after LAD ligation was measured by cardiac MRI at 1-3 days post injury (dpi, acute injury period) and 10-12 dpi (recovery period), respectively.

### Cardiomyocyte Isolation

Isolation of cardiomyocytes for culture. The Neomyts Cardiomyocyte isolation kit (Cellutron) was used to isolate cardiomyocytes from the fresh heart tissue (rat, mouse, and human) for cell culture, following the manufacturer's instructions. Rat and mouse cardiomyocytes were cultured on glass coated with fibronectin (10 micrograms per milliliter (μg/ml)). Human fetal cardiomyocytes were cultured on glass coated with laminin (20 μg/ml).

Isolation of intact cardiomyocytes for quantification: We used a previously validated fixation digestion method to isolate intact cardiomyocytes from fresh or frozen myocardium for quantification (Mollova, M., et al. “Cardiomyocyte proliferation in human heart growth” Proceedings of the National Academy of Sciences January 2013, 110 (4) 1446-1451; DOI: 10.1073/pnas.1214608110). The myocardium was cut into 1 cubic millimeter (mm³) sized tissue blocks followed by incubation with 3.7% formaldehyde at room temperature for 1.8 hours. After being washed with PBS 3 times to remove the formaldehyde, the fixed tissue blocks were digested in enzyme solution (Collagenase B, 3.6 mg/ml; Collagenase D, 4.8 mg/ml) at 37 degrees Celsius (° C.), with 10 revolutions per minute (rpm) overhead rotation. The digested cells were collected every 24 hours from the supernatant, and the undigested tissue was re-suspended with fresh enzyme solution until all of the tissue pieces were digested. All of the digested cells from the same heart were then combined and allowed to settle to the bottom of the tube. The cell pellet was collected for further study.

Primary cardiomyocyte culture experiments: Neonatal (mouse or rat) cardiomyocytes or human fetal cardiomyocytes were plated in 4-well chamber slides pre-coated with fibronectin (10 μg/ml) at a density of 200,000 cells/well and cultured with DMEM/high glucose (for mouse and rat cardiomyocytes) or IMDM (for fetal human cardiomyocytes) containing 10% fetal bovine serum (FBS) and recombinant neuregulin 1 (rNRG1, 100 nanograms per milliliter (ng/ml)) overnight. All treatments and BrdU (30 micromolar (μM)) were added on the next day.

Adenoviral gene transfer: To generate the Adv-GFP-Ect2 vector, the GFP-tagged full-length Ect2 (Su et al. Targeting of the RhoGEF Ect2 to the equatorial membrane controls cleavage furrow formation during cytokinesis. Dev Cell 21, 1104-1115 (2011)) was cloned into pAd/CMV/V5-DEST™ gateway vector (Thermo Fisher, Cat #: V49320) and adenoviruses were generated in 293A cells according to manufacturer's instructions. We used Adv-Ect2-GFP (MOI=2,000), transduction period day 1 to day 3 (transduction efficiency>80%), followed by culture without adenovirus (day 3 to day 5). The cells were fixed at day 5 for further study. To test the effect of adenoviral mediated Ect2 gene transduction in cardiomyocytes with Ect2 gene inactivation (isolated from the newborn (P2) Ect2^{flox} mice), we transfected cardiomyocytes with Adv-CMV-iCre. In combination, Adv-CMV-iCre (MOI=500) and Adv-Ect2-GFP (MOI=2,000) were added simultaneously to the culture from day 1 to day 3. For YAP1 gene transduction, we used Adv-YAP1-WT (MOI=200) or Adv-YAP1-S127A (MOI=200), transduction period (day 1 to day 3). The cells were collected on day 3 for examining Ect2 expression or immunofluorescence study. For TEAD1-myc gene transduction, we used Adv-TEAD1-myc (MOI=100), transduction period day 1 to day 3. The cells were fixed at day 3 for immunofluorescence study. The Adv-GFP (MOI=500) vector was used as control.

β-adrenergic receptor signaling pathway studies: Forskolin (10 μM), propranolol (10 μM), dobutamine (10 μM), or a combination of propranolol (10 μM) and dobutamine (10 μM) were added to the culture from day 1 to day 3. The cells were collected for examining gene expression or immunofluorescence microscopy at day 3.

Gene knockdown with siRNA: siRNA against TEAD1 (50 nanomolar (nM)) or TEAD2 (50 nM) was added to cultured neonatal rat cardiomyocytes from day 1 to day 3 following the vendor protocol. The cells were collected at day 3 for examining the Ect2 expression by real-time RT-PCR or quantifying cardiomyocyte binucleation by immunofluorescence microscopy. We cultured cardiomyocytes isolated from β_{1/2}-AR double knockout pups (DKO, P2), added siRNA against Ect2 (50 nM) to the culture from day 1 to day 3, followed by fixation at day 3.

Video microscopy to determine cleavage furrow regression and cytokinesis failure: Cardiomyocytes were isolated from neonatal rats (NRVM, P2) and cultured in NS Medium containing 50 nanograms per microliter (ng/μL) NRG-1 (R&D Systems) in fibronectin-coated 35 mm glass bottom dishes (Part No. P35G-2-14-C-Grid, MatTek Corporation) or 8-well chambered coverglass (Part No. 155409, Lab-Tekll) for 48 hours prior to live cell imaging. For imaging, cardiomyocytes were maintained in an environmental chamber (Tokai-HIT) fitted on the motorized stage (Prior) of an inverted Olympus IX81-ZDC autofocus drift-compensating microscope. Images were acquired at multiple positions every 30 minutes by a CCD camera (Hamamatsu) using an Olympus APON60XOTIRF objective, NA 1.49, together with differential interference contrast (DIC) components. Image acquisition and analysis were done using Slidebook™ 5.0 software.

Video microscopy to determine dynamic GFP-Ect2 localization in cycling cardiomyocytes: Neonatal rat cardiomyocytes (P2) were cultured in NS medium containing 10% FBS (Cellutron Life Technologies) for 3 hours to allow the cells to attach to the surface of an 8-well chambered cover glass coated with fibronectin (20 μg/ml). To remove unattached cells, the NS medium was changed carefully to DMEM/F12 (no phenol red) containing 5% FBS and adenovirus (Adv-Ect2-GFP, MOI=2,000; Adv-GFP, MOI=500). The chambered cover glass was mounted on an onstage incubator (Tokai Hit) providing a physiological environment (37° C., humidity, air mixture containing 5% CO2) on the motorized stage of a Nikon TiE microscope. Time-lapse imaging was performed for 72 hours. Images were acquired in 10-minute intervals by a CMOS camera (Andor Zyla) using a Nikon Plan Apo 60× oil objective, utilizing the Nikon perfect focus system (PFS) together with filter sets for observing GFP fluorescence. Image acquisition and analysis were done using Nikon NIS Elements 4.5 software.

ToF/PS myocardium tissue culture: Discarded myocardium samples were collected during ToF/PS surgery, cut into ˜1 mm³ tissue blocks, and cultured in IMDM media containing 10% FBS, recombinant neuregulin 1 (rNRG1, 100 ng/ml), and BrdU (30 μM) for 6 days. Samples were treated with forskolin (10 μM), propranolol (10 μM), dobutamine (10 μM), or a combination of propranolol (10 μM) and dobutamine (10 μM).

Quantification of cardlomyocyte endowment (number of cardiomyocytes) of mouse hearts

Counting of cardiomyocytes after fixation and digestion: All fractions from the digestion of a heart were combined in 2 milliliters (mL) PBS and cardiomyocytes were quantified using a hemocytometer.

Quantification of cardiomyocytes with unbiased stereology: Mouse hearts were washed in cardioplegia solution (PBS, 25 μM potassium chloride (KCl)), weighed, and fixed with 3.7% formaldehyde at room temperature overnight. The hearts were then placed in 30% sucrose at 4° C. for 48 hours. The atria were cut off and the ventricles were embedded in OCT and sectioned on a Leica CM1950 cryostat in cross-sectional orientation (thickness 15 μm), resulting in 75-80 slides/heart with 4 sections each. For random systematic sampling we used a random number generator ranging between 1-6 to determine the first slide and then selected every 17^{th} slide for staining. Myocardial volume and scar were quantified by point count method on AFOG-stained sections. Briefly, tissue sections were stained with Acid fuchsin-orange G (AFOG; Polizzotti et al.) and photomicrographs were taken on a Leica MZ26 dissector microscope (objective lens 10×). Areas of myocardium (red after AFOG staining) and scar (blue after AFOG staining) were quantified using ImageJ (version 1.51s). The image was overlaid with a grid to determine area per point. The distance between selected sections was calculated (17^{th} slide×4 sections/slide×15 μm section thickness). The LV myocardial volume was measured by counting the number of grids on both LV myocardium and the scar region. We used the optical dissector method to determine the volume density of cardiomyocyte nuclei. The optical dissector operates optimally at 3 μm distance between lookup and counting frames. Adult mouse hearts were sectioned with a cryostat set at 15 μm. Slides were stained with α-actinin and Hoechst and imaged with an A1R laser-scanning confocal microscope (×60 oil lens).

Briefly, the immunofluorescence stained sections were imaged using a Nikon A1R confocal microscope. We selected four random spots on the technically-best section from each slide and analyzed 20 random samples from each heart. The total number of positive cardiomyocyte nuclei per heart was counted and the mean per sample volume was calculated. Total number of cardiomyocytes (endowment) was calculated as: Number of cardiomyocytes=Number of cardiomyocyte nuclei/(Mono %+2×Bi %=3×Tri %+4×Tetra %).

Quantification of cardiomyocyte size: To measure the size (2D) of cardiomyocytes in mouse pups with Ect2 inactivation (αMHCCre; Ect2^{F/F}, using αMHC-Cre;Ect2^{F/Wt} as control), cardiomyocytes were isolated at P1 using the fixation-digestion method, followed by immunostaining in suspension with α-actinin and pancadherin antibodies, and Hoechst for nuclei staining and imaging with a Nikon TiE microscope with Zyla CMOS camera. Immunofluorescence photomicrographs were used to identify mono and binucleated cardiomyocytes and bright-field photomicrographs were used to measure the area of single cardiomyocytes using ImageJ software. The distribution of cell size was analyzed using GraphPad Prism software.

Quantification of Mouse Heart Function

Adult mouse cardiac MRI: Mice were anesthetized with 4% isoflurane mixed with room air in an induction box for 1 to 3 minutes. The depth of anesthesia was monitored by toe reflex, extension of limbs, and spine positioning. Once an appropriate depth of anesthesia was established, mice were placed on a custom-built mouse holder and the anesthesia was maintained by 1.5 to 2% isoflurane with 100% oxygen via a nose cone. Respiration was continuously monitored by placing a small pneumatic pillow under the animal's diaphragm which was connected to a magnet-comparable pressure transducer feeding to a physiological monitoring computer equipped with respiration-waveform measuring software (SA Instruments, Stony Brook, N.Y.). The respiration waveform was automatically processed to detect inspiration, expiration, and respiration rate. In-vivo cardiac MRI (CMR) was carried out on a Bruker Biospec 7T/30 system (Bruker Biospin MRI, Billerica, Mass.) with the 35-mm quadrature coil for both transmission and reception. Free-breathing-no-gating cine MRI with retrospective navigators was acquired with the Bruker Intragate module. For late-gadolinium enhancement (LGE) to quantify myocardial infarct size, Multihance (Gadobenate dimeglumine (GD), 529 mg/ml, Bracco Diagnostics, Inc, N.J., 0.1 millimole GD per kilogram (mmol Gd/kg) bodyweight, subcutaneous injection (s.c.)) was administered before the CMR acquisition. T1-weighted images to highlight LGE were acquired 15-20 minutes after the subcutaneous administration of Multihance. Eight T1-weighted short-axis imaging planes covering the whole ventricular volume with no gaps were acquired with the following parameters: Field of view (FOV)=2.5 cm×2.5 cm, slice thickness=1 mm, in-plane resolution=0.97 μm, flip angle (FA)=10 degrees, echo time (TE)=3.059 msec (millisecond), repetition time (TR)=5.653 msec. For cine CMR to determine cardiac function, white-blood cine movies with 20 cardiac phases were acquired with equivalent temporal resolution for the cine loops (16.5-21.5 msec per frame). Eight short-axis imaging planes covering the whole ventricular volume with no gaps and one long-axis plane were acquired with the following parameters: Field of view (FOV)=2.5 cm×2.5 cm, slice thickness=1 mm, inplane resolution=0.97 μm, flip angle (FA)=30 degrees, echo time (TE)=1.872 msec, repetition time (TR)=38.293 msec. To obtain the proportion of myocardial infarction (MI), we measured the angle of the portion of the myocardium displaying hyperintensity in the left ventricle wall of each scanned slice and divided the angle by 360° to get the percentage of infarction of the slice. The infarction of each scanned slice was then averaged to calculate the proportion of myocardial infarction. To quantify cardiac function from cine CMR, the left ventricular endocardial and epicardial boundaries of each imaging slice at the end-systole (ES) and the diastole (ED) were manually traced by a blinded researcher using the Paravision 5.1 Xtip software (Bruker Biospin MRI, Billerica, Mass.) to calculate the following functional parameters: left ventricular blood volume (LVV), left ventricular wall volume (LV wall), LV mass, stroke volume (SV), ejection fraction (EF), heart rate (HR), cardiac output (CO), longitudinal shortening, and radial shortening. LVV is calculated by summation of all short-axis slices. The ejection fraction (EF) was calculated using the following equation:

$$EF = \frac{{Σ}_{i} {A}_{i}^{ed} {h}_{i} - {Σ}_{i} {A}_{i}^{es} {h}_{i}}{{Σ}_{i} {A}_{i}^{ed} {h}_{i}} \times 100 \%$$

where Aᵢ^{es} is the internal left ventricle area of slice i at end systole, Aᵢ^{ed} the internal left ventricle area of slice i at end diastole, and hᵢ is the thickness of each scanned slice. The proportion of left ventricle wall thinning caused by adverse remodeling was calculated by measuring the proportion of the angle of the thinned left ventricle. To calculate the left ventricle wall thickening, we first selected the scanned slice that demonstrated the largest LGE portion. The thickness at both ends and the middle of the LGE portion was measured at end systole (d^{es}) and diastole (d^{ed}) and calculated the average. The left ventricle wall thickening was calculated through equation:

$$LV wall thickening = \frac{{d}^{es} - {d}^{ed}}{{d}^{ed}} \times 100 \% .$$

Neonatal mouse echocaidiography. We performed echocardiography using a Vevo 770 device (VisualSonics) with a 25 MHz probe (RMV-710B). Two-dimensional (2D) B-mode recordings covering both ventricles were obtained in the left parastemal short axis view. The left ventricular endocardium and epicardium boundaries at end-systole and diastole were manually traced by a blinded operator on the ImageJ software. The ejection fraction (JK) was calculated using the following equation:

$$EF = \frac{{A}^{ed} - {A}^{es}}{{A}^{ed}} \times 100 \%$$

where PQR is the left ventricular blood area at end systole, PQS is the left ventricular blood area at end diastole.

Single cell transcriptional profiling and analysis of mouse cardiomyocytes. Freshly isolated cardiomyocytes from fetal (E14.5) and neonatal (P5) transgenic mAG-hGem mice were sorted on a FACSAria (20 psi, 100 μm nozzle, Becton Dickenson Biosciences). The cycling cells (mAG+) were separated from non-cycling cells (mAG−) based on the fluorescent signals. Cardiac cells expressing mAGhGeminin transgene (mAG+) were identified using a sequential gating strategy. The cells were subjected to FACS, for isolation of single cycling and non-cycling cardiac cells. The population of mAG-hGem-positive cells that were in the cycle was determined by analyzing DNA contents after staining with Hoechst and RT-PCR with primers for cell cycle genes. Cells were gated sequentially for size, doublet exclusion, viability, and mAG-hGem expression. Cycling mAG-hGem-positive cells, non-cycling mAG-hGem-negative cells, and a merge of cycling mAG-hGem-positive cells and non-cycling mAG-hGem-negative cells determined that mAG-hGem-positive cells have higher DNA content.

Initial size gates for forward scatter (FSC) vs. side scatter (SSC) were set to select the large cardiomyocytes corresponding to larger and more granular cells. Cell doublet discrimination was performed by a combination of high forward scatter height and area FSC-H/FSC-A and SSC-H vs. SSC-W plots. Live cells were selected by 7-aminoactinomycin D (7AAD, 1 μg/mL final concentration, Invitrogen) live/dead cell distinction staining. Finally, live 7AAD negative cells were distinguished by their mAG fluorescence intensity using the FACSAria 488-nm excitation laser. The mAG+ and mAG− cell fractions were collected separately for further downstream analyses. FACSDiva Software was used for data acquisition and analysis. The cycling cardiomyocytes (mAG-positive) were sorted into 96-well plates containing reverse transcriptase buffer for the following linear amplification. Cardiomyocytes were identified by examining the expression of cardiomyocyte specific gene, Tnnt2, and non-cardiomyocyte contamination was identified and excluded by examining the expression of Pdgfrb gene.

Human cardiomyocytes: Freshly isolated single cardiomyocytes from human myocardium collected during surgery (ToF/PS) and abortion (fetal) were FAC sorted into 96-well plates containing reverse transcriptase buffer for transcriptional profiling and RNA sequencing. Following the synthesis of the first strand of cDNA, the molecular identity of the collected cardiomyocytes was confirmed by PCR for positive expression of the cardiomyocyte-specific gene Tnnt2 and negative expression of the non-cardiomyocyte gene Pdgfrb. After 2 rounds of linear amplification (in vitro transcription), the RNA samples were sequenced using HiSeq 2500 (Illumina).

Gene expression analysis: To analyze the data of RNA sequencing, we first trimmed reads for adapters and poly(A) contamination using trimmomatic (Bolger et al. Trimmomatic: a flexible trimmer for Illumina sequence data. Bioinformatics 30, 2114-2120 (2014)). We then mapped the trimmed pair-end reads to human/mouse genome build hg19 using HISAT2 (Kim et al. HISAT: a fast spliced aligner with low memory requirements. Nat Methods 12, 357-360 (2015)). Using human/mouse gene annotations (.gtf) downloaded from iGenomes, we assigned read counts to genes using HTSeq and htseq-count (Anders et al. HTSeq-a Python framework to work with highthroughput sequencing data. Bioinformatics 31, 166-169 (2015). Only exonic reads were counted with overlap assigned using the intersection non-empty method. We then further filtered all human samples by removing those with less than 60% overall reads mapping ratio and less than 1,000 expressed genes. The remaining samples were used for further analysis. Finally, to mitigate the difference between samples (e.g. reads counts difference due to variable sequencing depth), we normalized the samples using the housekeeping genes. We started by normalizing the gene expression (exonic reads count) to total mapped reads in the sample. Next, we obtained a list of housekeeping genes with most stable expression in heart from the study as reference genes. (Li et al. Selection of reference genes for gene expression studies in heart failure for left and right ventricles. Gene 620, 30-35 (2017)). For each sample, we calculated the geometric mean of those reference genes. Then, we calculated the average of the geometric mean across all samples. This average was further divided by the geometric mean of the reference genes in each sample to get a sample-specific normalization factor. Multiplying the gene expression counts by the lane-specific normalization factor, we get the normalized expression. Lastly, we converted the normalized expression into log 2 space.

Reverse transcription and real-time RT-PCR: The mRNA was extracted from cardiac tissue samples using TRIzol reagent (Life Technologies) following the protocol provided by the vendor. Briefly, the samples were first homogenized using a mortar and pestle. The TRizol reagent (1 mL) was added for each homogenized sample. HomogenizedFsamplesinTRzol reagentwere incubated at room temperature for 5 minutes, after which 0.2 mL of chloroform was added to each 1 mL of TRizol. The mixtures were shaken by hand for 15 seconds and then incubated for 2-3 minutes at room temperature. The mixtures were then centrifuged at 12,000×g for 15 minutes at 4° C. The clear upper aqueous phase containing the isolated RNA was carefully removed. The RNA was purified using the Qiagen RNeasy Plus Mini kit per the manufacturer's instructions. The purified RNA was then quantified on a Nanodrop. Reverse transcription was performed on the purified RNA using the BioRad iScript cDNA synthesis kit (Catalog #1708890). The kit was used according to the vendor protocol. The primers are listed in Table 1, below.

TABLE 1
PCR Primers and 5′-3′ Oligonucleotides

| | | |
|---|---|---|
| Gene | Forward | Reverse |

mRNA

| | | |
|---|---|---|
| Mouse | 5′-AGAAGGTGCTGGACATCCGAGA-3′ | 5′-CCTTTGGAAGCTCCTCTGACGT-3′ |
| Ect2 | (SEQ ID NO: 8) | (SEQ ID NO: 9) |
| Mouse | 5′-CATTGCTGACAGGATGCAGAAGG-3′ | 5′-TGCTGGAAGGTGGACAGTGAGG-3′ |
| β-actin | (SEQ ID NO: 10) | (SEQ ID NO: 11) |
| Mouse | 5′-CATCACTGCCACCCAGAAGACTG-3 | 5′-ATGCCAGTGAGCTTCCCGTTCAG-3′ |
| GAPDH | (SEQ ID NO: 12) | (SEQ ID NO: 13) |
| Rat Ect2 | 5′-CGCAAGGAGAAAAGTTTAGGG-3′ | 5′-CATCCTGGCCTCATAATTGG-3′ |
| | (SEQ ID NO: 14) | (SEQ ID NO: 15) |
| Rat | 5′-TTCAAAATGAAGAAACGAGAAAAG-3′ | 5′-AGGGCCATCGATGAACTGT-3′ |
| Racgap1 | (SEQ ID NO: 16) | (SEQ ID NO: 17) |
| Rat | 5′-TGGACCGATGTAGAAACAAGG-3′ | 5′-GTTGTGCGTGGTGCTGAT-3′ |
| MKLP1 | (SEQ ID NO: 18) | (SEQ ID NO: 19) |
| Rat | 5′-CGGATCGAATCCCTCACTC-3′ | 5′-TCTCCTTTTCCCGGGTCTT-3′ |
| GEF-H1 | (SEQ ID NO: 20) | (SEQ ID NO: 21) |
| Rat β- | 5′-CCCGCGAGTACAACCTTCT-3′ | 5′-CGTCATCCATGGCGAACT-3′ |
| actin | (SEQ ID NO: 22) | (SEQ ID NO: 23) |
| Rat | 5′-GGCAAGTTCAACGGCACAGT-3′ | 5′-TGGTGAAGACGCCAGTAGACTC-3′ |
| GAPDH | (SEQ ID NO: 24) | (SEQ ID NO: 25) |

Genomic DNA

| | | |
|---|---|---|
| Mouse | 5′-CGTTTCCGACTTGAGTTGCC-3′ | 5′-ACTCGGGTGAGCATGTCTTT-3′ |
| Rosa26 | (SEQ ID NO: 26) | (SEQ ID NO: 27) |
| Mouse | 5′-TCCTCCGGGTG GACCAGAG-3′ | 5′-CTGGCTTCATAATTGGAG TGC-3′ |
| Ect2^{flox} | (SEQ ID NO: 28) | (SEQ ID NO: 29) |

TaqMan assay: qRT-PCR was performed using Fast Taqman reagents (Thermo-Fisher). Probes were obtained from Thermo-Scientific including CYR61 (Mm00487498_m1), CTGF (Mm01192933_g1), ECT2 (Mm00432964_m1), and 18S (4319413E). All reactions were performed using a 1:10 diluted cDNA while mRNA expression levels were estimated using the 2DDCt method.

Examination of TEAD-binding sites by Luciferase Assay

Construct preparation: To generate deletions of the Ect2-promoter region, we first subcloned the 2.8 kb Ect2 promoter region into the luciferase reporter construct pGL4.10 (Promega, Madison Wis.). Using this construct, we generated a deletion construct that removed all five TEADbinding sites and the DNA between them (Ect2-Promoter−Δ_{2kb}) and a construct that only removed the nucleotides of the binding sites (Ect2-Promoter−Δ₁₋₅). All plasmid constructs were amplified using TOP-10 competent bacteria and plasmid DNA was isolated and purified using the Qiagen Midi-Prep System (Qiagen, Germany) according to the manufacturer's instructions.

Once purified, the plasmid constructs were sequence-verified to eliminate the possibility of unwanted mutations.

Transfection into HEK-293 cells: To measure the ability of the Ect2-promoter regions to generate a luciferase signal, we transfected HEK-293 cells with the following plasmids: 1) wt-Ect2-promoter-pGL4.10; 2) Ect2-Promoter−Δ_{2kb}−pGL4.10; 3) Ect2-Promoter−Δ₁₋₅-pGL4.10;4) Empty—pGL4.10; 5) pGL3.1-Renilla-Control. 4 micrograms (μg) of plasmid DNA were transfected into one well of a 6-well plate of HEK-293 cells using the lipofectamine 2,000 system (Invitrogen, Calif.) according to the manufacturer's instructions. Each pGL4.10 group was transfected along with an equal amount of the pGL3.10-Renilla-Control vector. Following transfection, the cells were allowed to incubate in a standard tissue culture incubator for 48 hours to allow for optimal luciferase construct expression.

Quantification of Ect2 promoter activity: To perform the luciferase measurements on cells transfected with Ect2-promoter deletions, we used the Dual Luciferase Reporter Assay System #E1910 (Promega, Madison, Wis.). After 48 hours of incubation, the media was removed and the cells were washed 2 times with PBS. Then the cells were lysed through incubation in the supplied passive lysis buffer for 20 min. After lysis, the mixture was centrifuged at 13,000×g for 5 minutes to pellet the debris, and the cell lysate was collected for subsequent analysis. The luciferase activity in each group was quantified according to the manufacturer's instructions. We prepared solutions of the Stop-n-Glo reagent and the Luciferase Assay II reagent (LAR2) as described in the manufacturer's instructions. The prepared solution was then loaded into the injectors of a Synergy H1 Hybrid plate reader (Biotek, Winooski, Vt.), and the first 100 μl of the solution was dispensed. We then placed 20 μl of cell lysate into the bottom of a Greiner Cellstar™ microclear bottom 96-well plate (Sigma, St. Louis, Mo.) and loaded it into the plate reader. 100 μl of LAR2 was then dispensed into 1 well and measurement of firefly luciferase was taken over a period of 10 sec. After measurement, 100 μl of Stop-n-Glo reagent was injected into the same well and incubated for 5 sec to quench the firefly luciferase reaction. The measurement of Renilla-control luciferase was performed for 10 seconds to measure the background luminescence activity. The measurement was repeated for each well. The difference between luminescence obtained from the experimental firefly luciferase and the Renilla luciferase measurements was calculated as the activity of the Ect2 promoter. The cell lysate from each transfection was transferred to 10 wells of a 96-well plate. The values of the activity of the transfected Ect2 promoter were averaged among the 10 wells.

### Immunofluorescence Microcopy

Cardiomyocytes cultured on glass surfaces: Cultured cardiomyocytes were fixed with 3.7% formaldehyde or 4% paraformaldehyde at room temperature for 12 minutes. After being washed in PBS 3 times, the cells were immersed in permeabilizing and blocking solution (0.5% Triton X-100, 5% donkey or goat serum in PBS) for 30 minutes. Then, the mixture of primary antibodies was added to the samples and incubated overnight at 4° C. After being washed 3 times in PBS, the cells were immersed in a mixture of secondary antibodies and incubated at room temperature for 1 hour. The nuclei were counterstained with Hoechst 33342 (Invitrogen, dilution 1:1000) at room temperature for 5 minutes. Then, the samples were dipped into distilled water for 15 seconds. The cells were mounted in 10 μl mounting media containing 1% N-propylgallate dissolved in glycerol and sealed with nail polish. To determine the activity of RhoA (RhoA-GTP) at the midbody, the cells were fixed in 0.5 ml ice-cold trichloroacetic acid (TCA) solution (10% w/v) for 15 minutes and then washed 3 times with PBS containing 30 mM glycine (Hayashi et al. Immunofluorescence detection of ezrin/radixin/moesin (ERM) proteins with their carboxyl-terminal threonine phosphorylated in cultured cells and tissues. J Cell Sci 112 (Pt 8), 1149-1158 (1999); Nishimura et al. Centralspindlin regulates ECT2 and RhoA accumulation at the equatorial cortex during cytokinesis. Journal of cell science 119, 104-114 (2006)). The cells were incubated in an ice-cold mixture of permeabilizing and blocking solution (5% goat serum, Triton X-100 0.5 μl/ml in PBS) for 60 minutes. Primary antibodies (RhoA-GTP, mouse IgM, New East Biosci, 26904, dilution 1:100); α-actinin (mouse IgG1, Sigma A7811, dilution 1:200); Aurora B kinase (rabbit, Abcam, ab2254, dilution 1:200). Secondary antibodies (goat anti-mouse IgG1 633, ThermoFisher A-21126, dilution 1:200; goat anti-mouse IgM 594, ThermoFisher A-21044, dilution 1:200; goat anti-rabbit 488, ThermoFisher A-11034, dilution 1:200).

Fixation-digested cardiomyocytes in suspension: For BrdU staining, the isolated cardiomyocytes were first incubated in 2N HCl solution containing Triton X-100 (Fisher Scientific, BP 151-500) at room temperature for 60 minutes. The HCl was then neutralized with the same amount of 2N NaOH solution. The isolated cardiomyocytes were then blocked in a solution of 5% goat serum (Sigma, G9023-10 ML), or donkey serum (Sigma, D9963-10ML), and incubated in primary antibodies at 4° C. overnight. After PBS wash, the cells were incubated in mixture of secondary antibodies at 4° C. overnight. The cells were washed with PBS, and incubated in Hoechst solution (PBS, 1:1,000) at room temperature for 5 minutes followed by three rounds of PBS wash. The cells were then ready for future study.

Heart sections: Hearts were resected, washed in PBS containing 25 μM KCl, and fixed immediately in 3.7% formaldehyde at room temperature for 8 hours. The fixed hearts were washed in PBS and immersed in 30% sucrose solution at 4° C. for 24 hours. After being embedded in optimum cutting temperature (OCT) compound, the frozen block was trimmed and the exposed heart tissue was cut into 15 μm thick sections with a Leica CM1950 cryostat and adhered to glass slides (Color Frost, Fisher). The slides were fixed in 3.7% formaldehyde, permeabilized in 0.5% Triton X-100, and blocked in PBS containing 20% goat serum and 0.2% Tween 20. Then, the mixture of primary antibodies was added to the samples and incubated at 4° C. overnight. After being washed 3 times in PBS, the cells were immersed in a mixture of secondary antibodies and incubated at room temperature for 1 hour. The nuclei were stained with Hoechst 33342 (Invitrogen, dilution 1:1,000) at room temperature for 5 minutes. Then, the samples were dipped into distilled water for 15 seconds. The cells were mounted in 10 μl mounting media containing 1% N-propyl-gallate dissolved in glycerol and sealed with nail polish.

### Quantification of Animal Cardiomyocyte Cell Cycle Activity

Cardiomyocytes cultured on glass surfaces: To assess S-phase, BrdU (30 μM) was added to the culture medium. BrdU (antibody verification) and cardiomyocyte markers (Troponin I, α-actinin) were stained by immunofluorescence. Neonatal rat ventricular cardiomyocytes (NRVM, Sprague-Dawley, isolated on P2) were cultured with or without BrdU (30 μM). After formaldehyde fixation and DNA denaturation with hydrochloric acid (HCl) (2N, 60 minutes at room temperature), the cells were treated with or without primary BrdU antibody (Abcam, generated from rat, 1:200), which was followed by incubation with fluorophore-conjugated secondary antibody. Troponin I antibody and Hoechst were used to identify cardiomyocytes and nuclei, respectively. To assess M-phase, phospho-Histone H3 (H3P) and cardiomyocyte markers (Troponin I, α-actinin) were stained. Both BrdU-positive and -negative cardiomyocytes, both H3P-positive and -negative cardiomyocytes were counted with a Nikon A1R confocal microscope. Then, the percentage of BrdU-positive and H3P-positive cardiomyocytes was calculated.

Intact cardiomyocytes in suspension. To assess M-phase, intact cardiomyocytes were isolated by fixation-digestion. The solution containing the stained cells was then transferred to 8-well chambered cover glass (400 μl/well). After the cells were settled down to the bottom of the chambers, the number of mono/bi/multinucleated cardiomyocytes were counted with a fluorescence microscope, and the percentage of mono/bi/multinucleated cardiomyocytes was calculated. To quantify the proportion of H3P-positive cardiomyocytes, the concentration of stained cardiomyocytes in suspension (C_{cardiomyocytes}) was first measured using a hemocytometer. The cell suspension (400 μl) was then transferred to a well of a chamber slide, and the total number of H3P-positive cardiomyocytes (H_{H3P+}) inside the well was counted with a TiE epifluorescence microscope. The proportion of the H3P-positive cardiomyocytes (P_{H3P+}) was calculated using the following formula:

$${H}_{H 3 P +} = \frac{{N}_{H 3 P +}}{400 μL \times {C}_{cardiomyocytes}} \times 100 \% .$$

Heart sections: To determine S-phase, BrdU (40 μg/g body) was administered to P7 ICR mice and the hearts were resected at P8, fixed, and cryosectioned at 15 μm thickness. From each heart, four heart sections at a distance of 300 μm were selected and imaged by tile scanning with a Nikon A1R confocal microscope. The total number of BrdU-positive cardiomyocytes at the selected heart sections was counted. The total area of the selected heart sections was measured by Fiji software. The density of BrdU-positive cardiomyocytes (cells/mm²) in the hearts was calculated using the following equation:

$$Density of {BrdU}^{+} = \frac{{Σ}_{4 sections} {BrdU}^{+} CMs}{{Σ}_{4 sections} Heart Section Area} .$$

¹⁵N-thymidine labeling of human cardiomyocytes in vivo and analysis: The clinical study protocol was approved by the IRB, and informed consent was obtained from the parents. Based on prior human Multi-isotope Imaging Mass Spectrometry (MIMS) studies (Steinhauser et al. Multi-isotope imaging mass spectrometry quantifies stem cell division and metabolism. Nature 481, 516-519 (2012); Guillermier et al. Imaging mass spectrometry demonstrates age-related decline in human adipose plasticity. JCI Insight 2, e90349 (2017)), the patient received ¹⁵N-thymidine (50 mg/kg p.o., Cambridge Isotope Laboratories, on five consecutive days) at 3.5 weeks of age. The patient underwent surgery at 6 months of age. A discarded piece of right ventricular myocardium was obtained, fixed in 4% paraformaldehyde, embedded in LR White, and 500 nm sections were mounted on silicon chips. Multiple isotope imaging mass spectrometry (MIMS) was performed on myocardial sections utilizing the NanoSIMS 50L (CAMECA) and previously described analytical methods (Senyo et al. Mammalian heart renewal by preexisting cardiomyocytes. Nature 493, 433-436 (2013)). ¹⁵N-thymidine labeling was measured by quantification of the ¹²C¹⁵N—/¹²C¹⁴N-ratio, obtained in parallel with mass images utilized for histological identification (¹²C¹⁴N—, ³¹P—, ³²S—). Quantitative mass images were then analyzed using OpenMIMS version 3.0 (https://github.com/BWHCNI/OpenMIMS74), a customized plugin to ImageJ (NIH). An observer blinded to the ratio images identified nuclei and assigned cellular identity using the ¹⁴N (¹²C¹⁴N—), ³¹P—, and³²S images as previously described (Senyo et al.). Cardiomyocyte nuclei were identified by their close association with sarcomeric structures. The total number of both ¹⁵N-thymidine-positive and -negative cardiomyocytes in both mono- and bi-nucleated cardiomyocytes were counted. The percentage of ¹⁵N-thymidine-positive cardiomyocytes in both mono- and bi-nucleated cardiomyocytes were calculated.

### Quantification of Mono- and Binucleated Cardiomyocytes

Intact cardiomyocytes in suspension. Cardiomyocytes were isolated from fresh or frozen (human or mouse) ventricle myocardium using the fixation-digestion method, followed by immunostaining as described above. The cells were then imaged and quantified with a Nikon A1R microscope for mono/bi/multi-nucleation.

Cardiomyocytes cultured on glass surfaces. Primary (mouse, rat, or human) cardiomyocytes were cultured with BrdU (30 μM) and fixed with 3.7% formaldehyde. The BrdU-positive nuclei, cell-cell boundaries (Pan-Cadherin antibody), and cardiomyocyte markers (α-actinin or troponin I antibody) were labeled by immunofluorescence. The stained cells were imaged under Nikon confocal microscope. The binucleated cardiomyocytes and all BrdU-positive cardiomyocytes were quantified using Fiji software.

Cultured ToF/PS myocardium. Cardiomyocytes were isolated from cultured myocardium with the fixation-digestion method. After denaturing the DNA with 2N HCl and neutralization with 2 Normal (N) sodium hydroxide (NaOH), immunofluorescence antibody labeling was performed in solution. The proportion of binucleated and BrdU-positive was quantified with a Nikon A1R microscope.

### Quantification of Ploidy of Cardiomyocyte Nuclei

Intact cardiomyocytes in suspension. The intact cardiomyocytes were isolated from frozen or fresh (human ToF/PS, or mouse with Ect2 inactivation) myocardial samples using the fixation digestion method. Then, the cardiomyocytes were labeled with α-actinin antibody, and the nuclei were stained with Hoechst. The stained cardiomyocytes were imaged using an epifluorescence microscope with a CMOS camera. The Hoechst fluorescence intensity of the nucleus of mononucleated cardiomyocytes was measured by ImageJ software after correction for background fluorescence. The fluorescence intensity the mononucleated cardiomyocyte nuclei was then normalized by that of the non-cardiomyocytes to obtain the ploidy of the cardiomyocytes.

Ploidy of ¹⁵N-thymidine positive cardiomyocyte nuclei: Adjacent sections of ¹⁵N-thymidine positive nuclei of interest were selected, fixed, and stained with Hoechst. The DNA contents of the nuclei in mononucleated cardiomyocytes were evaluated by measuring the Hoechst fluorescence intensity. Ploidy was determined by normalizing the measured DNA content by that of non-cardiomyocytes, after elimination of background fluorescence.

### Statistical Analyses

Statistical testing was performed with Student's t-test, Fisher's exact test, and ANOVA, followed by Bonferroni post hoc testing, as indicated. A two-sided P value≤0.05 was accepted as statistically significant. Statistical analyses were performed with GraphPad Prism, version 6.

### Results and Discussion

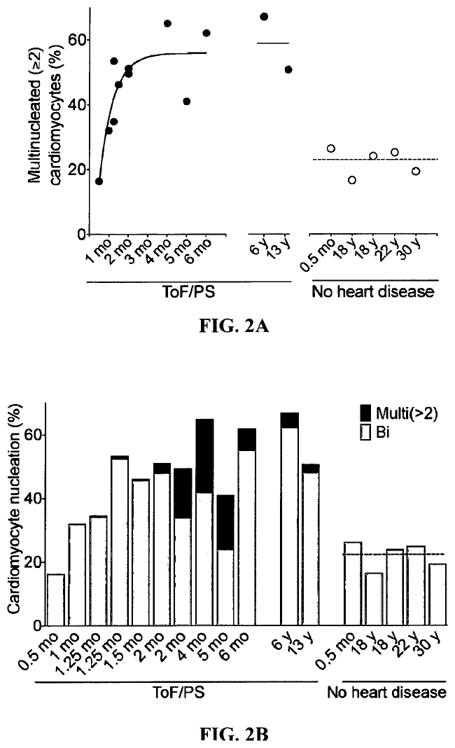

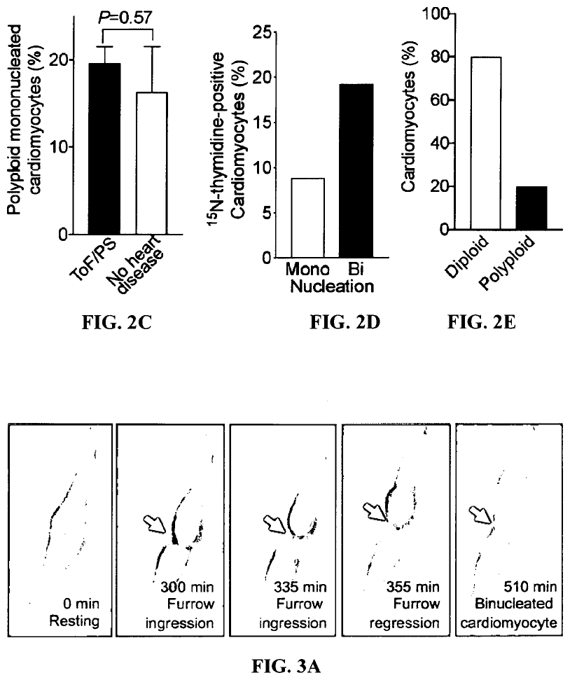

Infants with Tetralogy of Fallot (ToF/PS) generate a higher proportion of binucleated cardiomyocytes: Myocardium was analyzed by multiple-isotope imaging mass spectrometry (MIMS) at 7 months. The ¹⁵N/¹⁴N ratio reveals ¹⁵N-thymidine incorporation. We examined samples from the right ventricle of patients with ToF/PS and made the surprising observation that the percentage of binucleated cardiomyocytes was increased to 50-60% (FIG. 2A), suggesting increased cytokinesis failure. Temporal analysis revealed that newborns with ToF/PS showed the expected percentage of 20% binucleated cardiomyocytes, but that the increase happened in the first 6 months after birth (FIG. 2A). All ToF/PS patients>2 months had cardiomyocytes with >2 nuclei; this is a very rare phenotype in humans without heart disease (FIG. 2B), suggesting that multiple serial cytokinesis failures occurred. Bi- and multinucleated cardiomyocytes were present in a 6- and a 13-year-old ToF/PS patient, suggesting that bi- and multi-nucleated cardiomyocytes persist. As the proportion of polyploid mononucleated cardiomyocytes increases in humans after birth, we determined the ploidy of nuclei in mononucleated cardiomyocyte in ToF/PS infants and found that this was not different in comparison with published, age-matched controls (FIG. 2C). To directly assess the generation of mono- and binucleated cardiomyocytes in ToF/PS patients, we have taken a new research approach. We labeled a 1-month-old ToF/PS baby with ¹⁵Nthymidine and examined uptake and retention with multiple-isotope imaging mass spectrometry (MIMS) at 7 months of age. Twelve percent of cardiomyocytes were ¹⁵N-thymidine positive, indicating that these were generated between label administration at 1 month and removal of myocardium at 7 months after birth. Of mononucleated cardiomyocytes, 8.9% were ¹⁵N-thymidine positive (FIG. 2D). Of these, 80% had diploid nuclei (FIG. 2E), in agreement with the results shown in FIG. 2C. Since mononucleated cardiomyocytes make up approximately 45% of all cardiomyocytes in ToF/PS hearts at this age (see FIG. 2A), this shows that 0.089×0.8×0.45=3.2% of all cardiomyocytes were generated by division between 1 and 7 months after birth. Of binucleated cardiomyocytes, 19.2% were 15N-thymidine positive (FIG. 2D). Since binucleated cardiomyocytes make up the other 55%, this indicates that 0.192×0.55=10.6% of all cardiomyocytes were generated by cytokinesis failure in the period between 1 and 7 months after birth. Thus, cytokinesis failure was 3.3 times more common than division as a cell cycle outcome during this period, which explains the higher percentage of binucleated cardiomyocytes generated in hearts with ToF/PS. Prior research showed that an equal proportion of mono- and binucleated cardiomyocytes should be generated since their relative prevalence does not change in humans without heart disease (Mollova et al.; Bergmann et al.). These findings motivated us to determine the mechanisms controlling cytokinesis in cardiomyocytes.

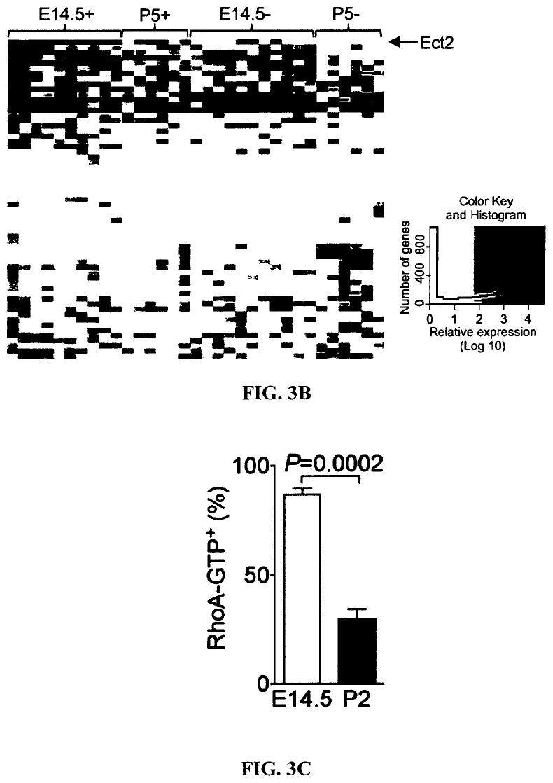

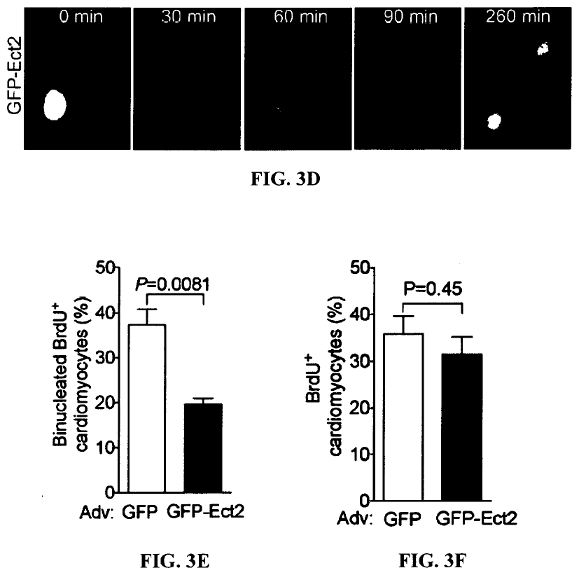

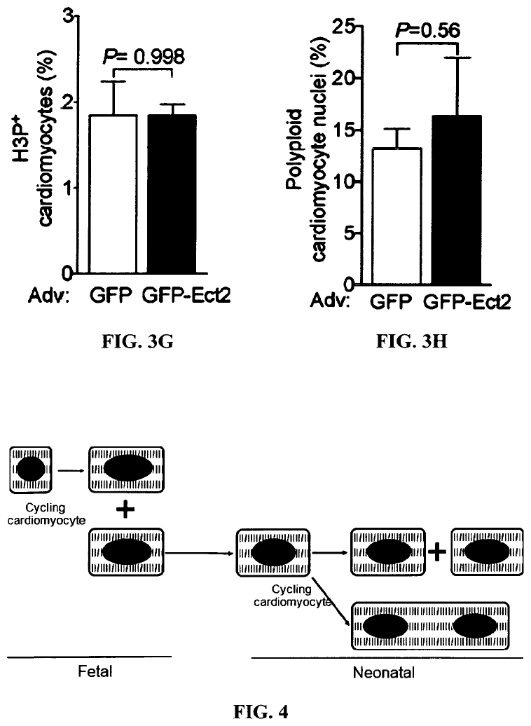

Cardiomyocyte cytokinesis failure is associated with low expression of Ect2: To determine the cellular mechanisms of cytokinesis failure in cardiomyocytes, we performed live cell imaging with neonatal rat ventricular cardiomyocytes that undergo binucleation (NRVM, FIG. 3A). Cleavage furrow ingression was observed in 80% of the cardiomyocytes studied, followed by cleavage furrow regression. We used a transgenic mouse model expressing the fluorescent ubiquitination-based cell cycle indicator (FUCCI) to highlight cell cycle progression; we noted normal cell cycle progression until cleavage furrow regression. This finding demonstrates that failure of abscission generates binucleates from mononucleated cardiomyocytes.

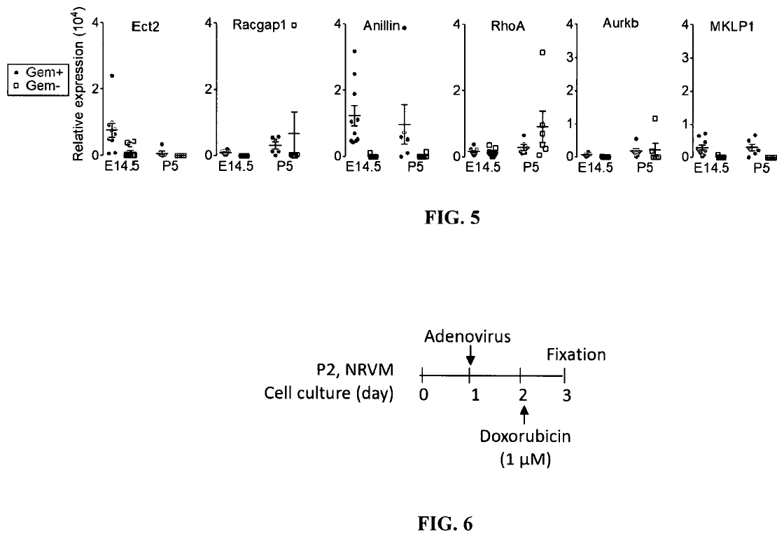

To identify the molecular mechanisms of cleavage furrow regression, we separated cycling from non-cycling cardiomyocytes and took a single cell transcriptional profiling approach to compare the expressed genes (FIG. 4). We isolated embryonic (Embryonic day 14.5, E14.5) and neonatal (Postnatal day 5, P5) cardiomyocytes, identified cycling cardiomyocytes with the mAG-hGem reporter of the FUCCI indicator, and separated them by FACS. We performed deep, genome-wide, single-cell transcriptional analysis with the Eberwine method (Dueck et al. Deep sequencing reveals cell-type-specific patterns of single-cell transcriptome variation. Genome biology 16,122 (2015)), followed by validation of the results. During cytokinesis, a contractile ring forms at the future division plane. Contraction of this ring is triggered by the cytokinesis protein ECT2, a RhoA guanine-nucleotide exchange factor. RhoA-GTP activates, via Rho-associated protein kinase (ROCK), non-muscle myosin II, which constricts the cleavage furrow. Because of the critical function of RhoA activation for cleavage furrow constriction, we examined the expression of Dbl-homology Rho-Guanine Nucleotide Exchange Factors (GEFs) in the single cell transcriptional dataset (FIG. 3B). Ect2 mRNA was present in cycling E14.5 cardiomyocytes but not in binudeating PS cardiomyocytes (FIG. 3B). Other genes controlling cytokinesis, i.e., Racgap1 (inactivating RhoA), RhoA, Anillin, Aurkb, and Mklp1, were present in P5 cycling cardiomyocytes (FIG. 5), indicating that Ect2 may be uniquely regulated. In accordance with the decreased Ect2 expression levels, RhoA activation (RhoA-GTP) was decreased in binucleating cardiomyocytes (FIG. 3C). Taken together, these results show insufficient Ect2 levels in cardiomyocytes lead to less RhoA activation, weakening their cleavage furrow constriction.

We tested whether increasing Ect2 expression enables cardiomyocyte abscission by expressing GFP-Ect2. Live cell imaging showed the functionality of GFP-ECT2 in cardiomyocytes (FIG. 3D). Transduction with GFP-Ect2 decreased the formation of binucleated cardiomyocytes 2-fold (FIG. 3E) without altering the proportion of cardiomyocytes in S- (measured by quantifying BrdU-positive cardiomyocytes, FIG. 3F) or M-phase (measured by quantifying phospho-histone H3-positive (H3P) cardiomyocytes, FIG. 3G), or inducing apoptosis (FIG. 6). In addition, expressing GFP-ECT2 did not alter the ploidy of cardiomyocyte nuclei (FIG. 3H). In conclusion, increasing Ect2 expression in cardiomyocytes has a specific positive effect on abscission without changing cell cycle entry or progression.

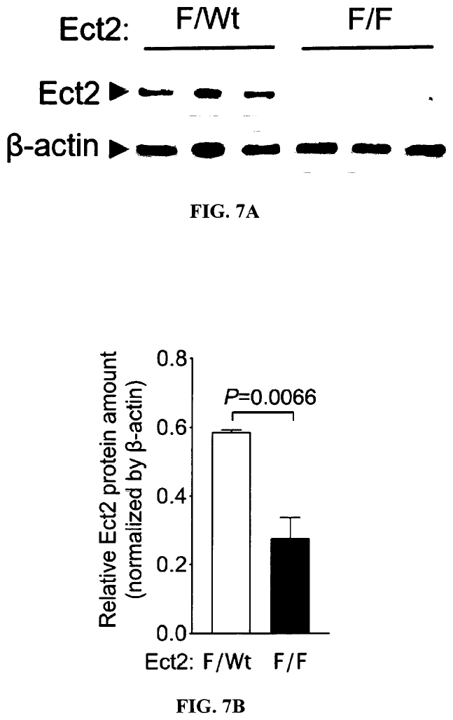

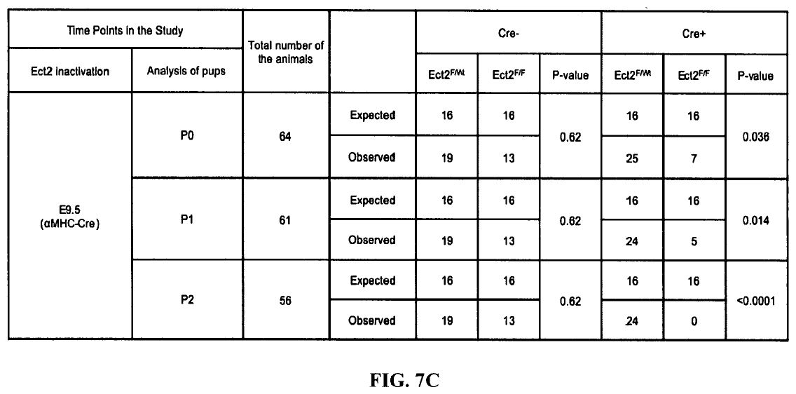

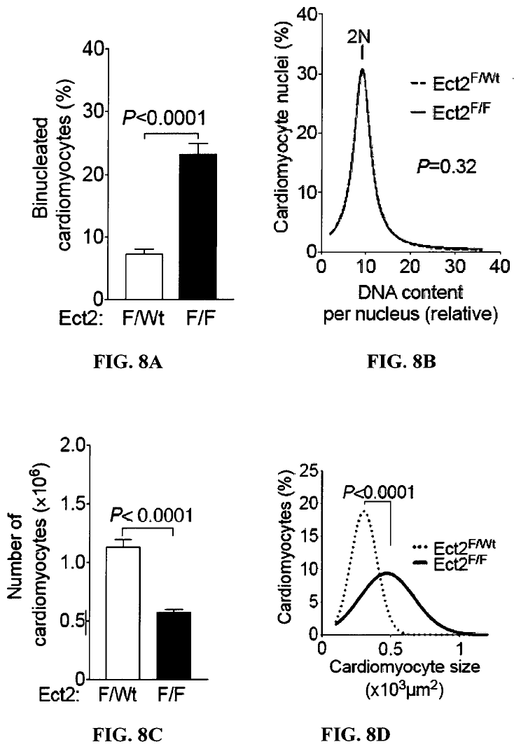

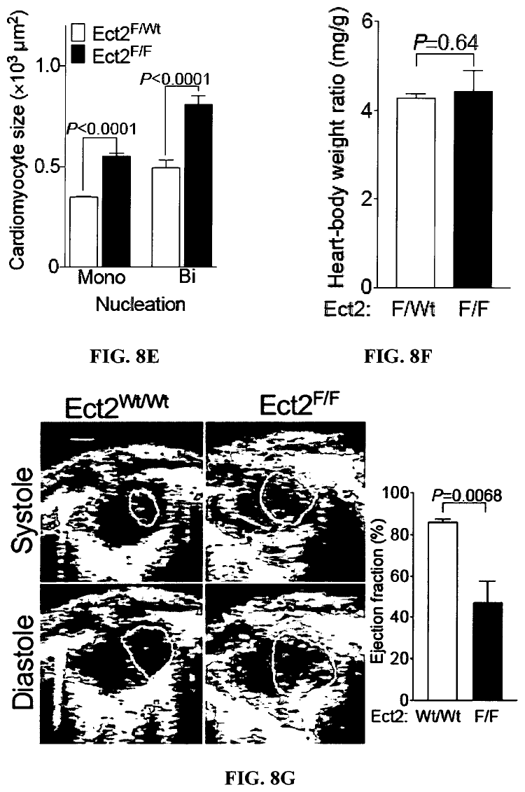

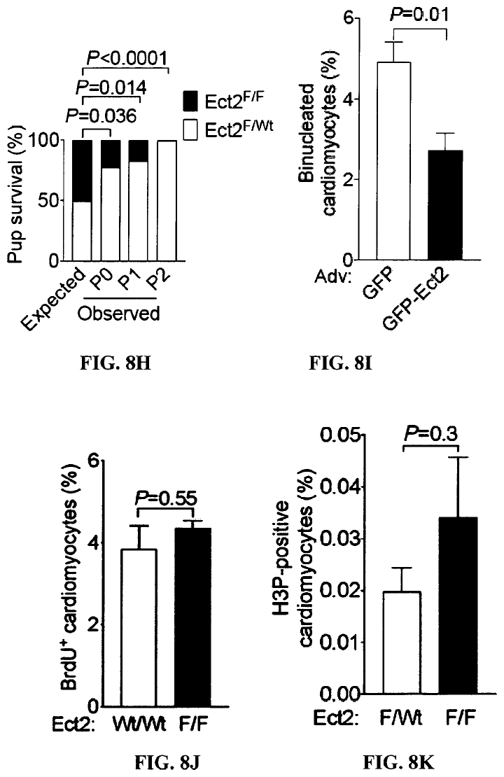

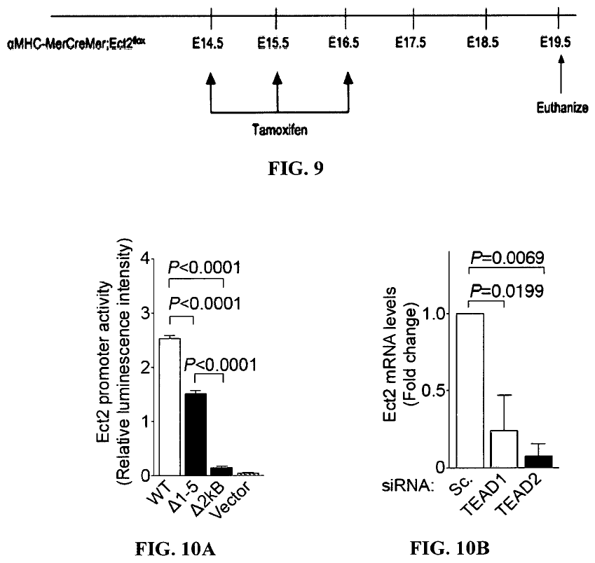

Lowering Ect2 expression reduces cardiomyocyte endowment and heart function: To determine the effect of lowering the expression of Ect2 in vivo, we inactivated the Ect2^{flox} gene in mice with αMHC-Cre (FIG. 7B). αMHC-Cre⁺; Ect2^{flox/flox} mice showed a 3.2-fold increase of binucleated cardiomyocytes (23.3%, FIG. 8A), compared to control (αMHC-Cre⁺;Ect2^{wt/flox}, 7.4%, P<0.0001), at P1. Ect2 inactivation did not change the DNA contents of nuclei (FIG. 8B). αMHC-Cre⁺;Ect2^{flox/flox} pups had 583,000±15,379 cardiomyocytes (n=5 hearts) at P1, a 49% decrease compared to αMHC-Cre⁺;Ect2^{wt/flox} mice (1,140,833±58,341 cardiomyocytes, n=12 hearts, P<0.0001, FIG. 8C). The mean cardiomyocyte size in αMHC-Cre⁺;Ect2^{flox/flox} mice was increased by 65% (FIG. 8D). Mono- and binucleated cardiomyocytes showed a similar increase of size (FIG. 8E). These results show that the lower number of cardiomyocytes (endowment; Botting et al. Early origins of heart disease: low birth weight and determinants of cardiomyocyte endowment. Clin Exp Pharmacol Physiol 39, 814-823 (2012)) in αMHCCre⁺;Ect2^{flox/flox} pups triggered hypertrophy in all working cardiomyocyte phenotypes, not only in the binucleated portion. The heart weight was unchanged (FIG. 8F). Echocardiography showed that αMHC-Cre⁺; Ect2^{flox/flox} had a significantly lower ejection fraction (EF=49.6%), compared with control (EF=85.9%, FIG. 8G), indicating decreased pump function. All αMHC-Cre⁺; Ect2^{flox/flox} pups died before P2 (FIG. 8H, FIG. 7C). To determine whether Ect2^{flox} gene inactivation alters cardiomyocyte cell cycle entry, we inactivated the Ect2^{flox} (Agah et al. Gene recombination in postmitotic cells. Targeted expression of Cre recombinase provokes cardiac-restricted, site-specific rearrangement in adult ventricular muscle in vivo. J Clin Invest 100, 169-179 (1997)) gene with αMHC-MerCreMer (Sohal et al. Temporally regulated and tissue-specific gene manipulations in the adult and embryonic heart using a tamoxifen-inducible Cre protein. Circ Res 89, 20-25 (2001)) using tamoxifen P0, 1, 2 in vivo, thus circumventing the lethality of inactivating with αMHC-Cre. We isolated cardiomyocytes at P2 and assessed genetic rescue with adenovirus-directed overexpression of Ect2, which reduced the formation of binucleated cardiomyocytes (FIG. 8I). Ect2^{flox} inactivation did not alter cell cycle entry (FIG. 8J), M-phase activity, as measured by quantification of H3P-positive nuclei (FIG. 8K), or induce apoptosis (FIG. 9). We identified binucleated αMHC-Cre⁺; Ect2^{flox/flox} cardiomyocytes with both nuclei being in M-phase, indicating that forcing cytokinesis failure does not prevent progression to karyokinesis in the next cell cycle in vivo (FIG. 8K). This finding suggests a mechanism for how cardiomyocytes with four and more nuclei could be generated, by serial cell cycle entry and progression to karyokinesis, followed by failure of abscission. Thus, Ect2 inactivation induced cytokinesis failure in cardiomyocytes, which decreased endowment by 50% and led to severely decreased ejection fraction and death.

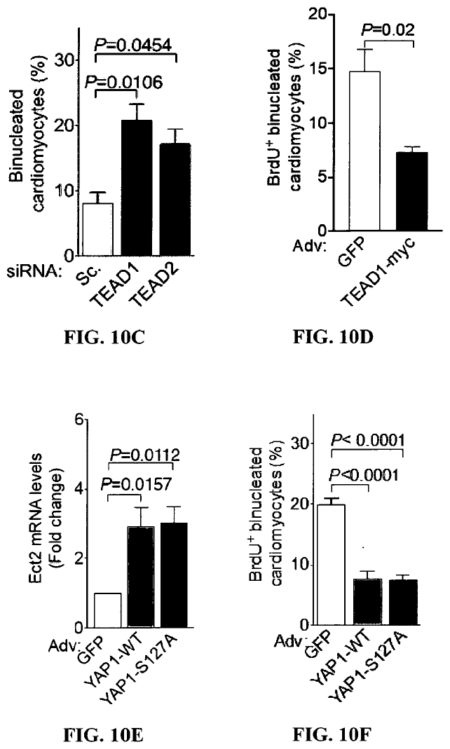

The Hippo tumor suppressor pathway regulates Ect2 gene transcription and cardiomyocyte division: We next sought to identify the mechanisms responsible for decreasing transcription of the Ect2 gene. YAP1, the central transcriptional co-regulator controlled by the Hippo pathway, forms a protein complex with TEAD transcription factors. We identified five binding sites for TEAD transcription factors in the Ect2 promoter. The wild type Ect2 promoter was modified to test the effect of the putative TEAD1/2-binding sites on the Ect2 promoter activity. Five putative TEAD1/2-binding sites were detected in the Ect2 promoter region through genome browser. All five putative TEAD-binding sites were removed from the Ect2 promoter and the continuous 2 kB DNA sequence containing all the five TEAD-binding sites was removed. Removing these TEAD-binding sites individually or en bloc decreased Ect2 promoter activity in luciferase assays (FIG. 10A). siRNA knockdown of TEAD1/2 reduced Ect2 mRNA levels (FIG. 10B) and increased the proportion of binucleated cardiomyocytes (FIG. 10C). Adenovirus-directed increase of TEAD1 expression decreased the formation of binucleated cardiomyocytes (FIG. 10D). Adenoviralmediated expression of wild type YAP1 (YAP1-WT) and a non-degradable version (YAP1-S127A) in NRVMs increased Ect2 mRNA levels (FIG. 10E) and reduced the proportion of binucleated cardiomyocytes (FIG. 10F). These results show that YAP1 and TEAD transcription factors regulate the expression of Ect2 and cardiomyocyte abscission.

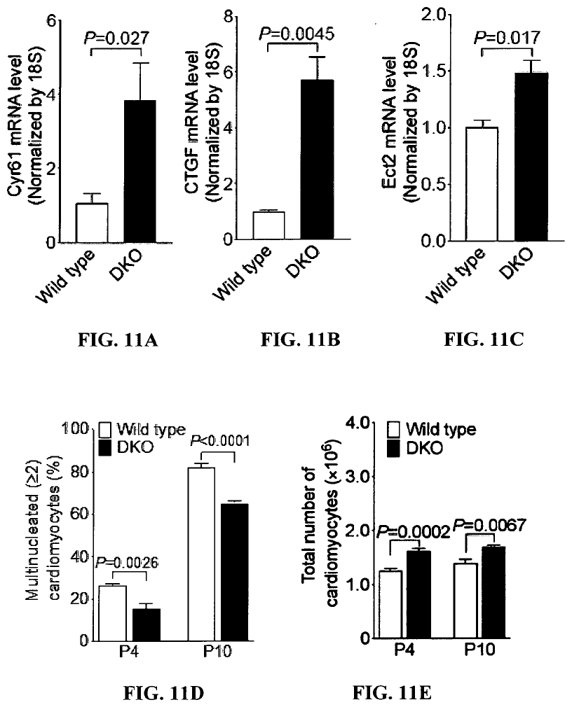

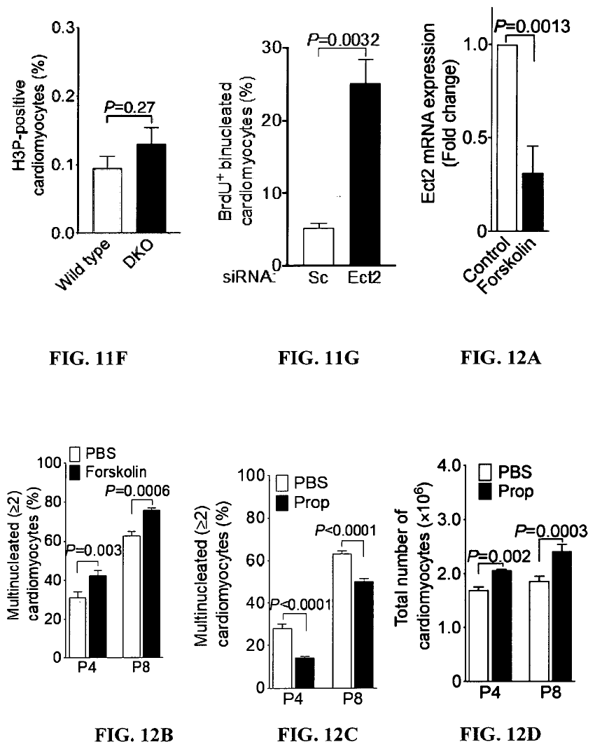

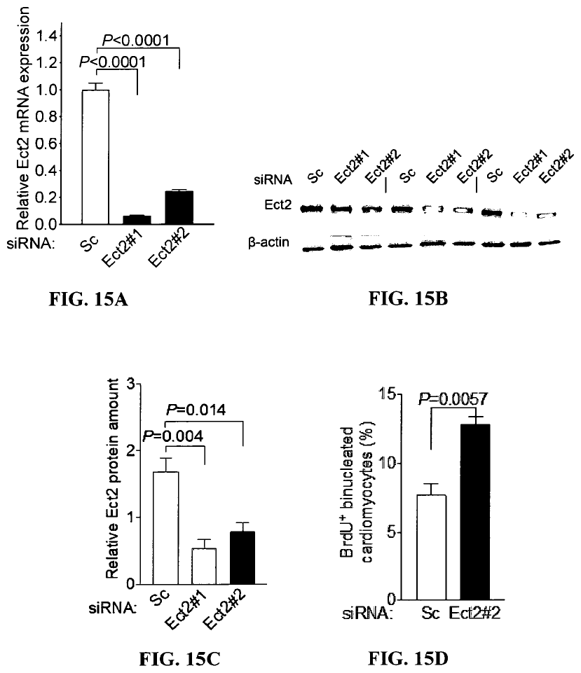

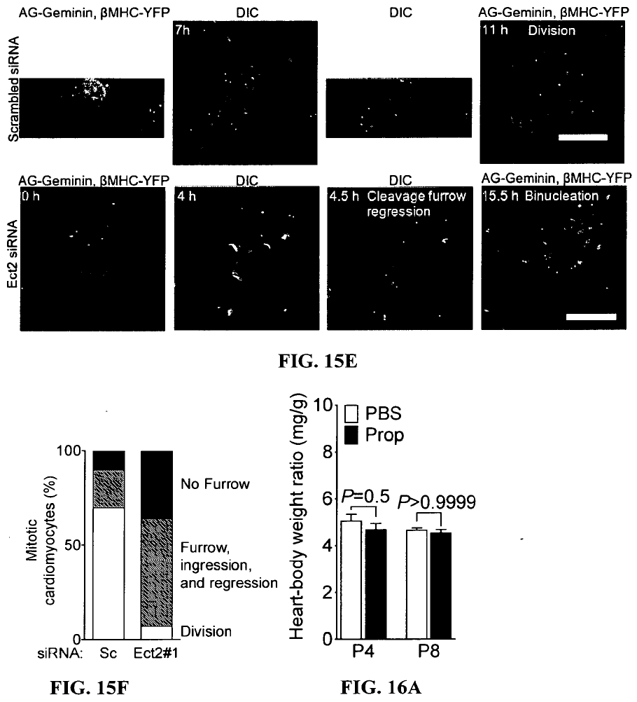

β-AR gene inactivation increases cardiomyocyte Ect2 expression, cytokinesis, and endowment: Because the Hippo pathway was shown to be activated by β-adrenergic receptor (β-AR) in the heart (F. X. Yu, F. X., et al., Regulation of the Hippo-YAP pathway by G-protein-coupled receptor signaling. Cell 150, 780-791 (2012)), we examined β₁-AR^{-/-}; β₂-AR^{-/-} (double-knockout, DKO) pups. Double-knockout pups showed increased transcription of the Hippo target genes Cyr61 (FIG. 11A) and CTGF (FIG. 11B), as well as Ect2 (FIG. 11C). These pups showed a lower proportion of binucleated cardiomyocytes (FIG. 11D) and a higher endowment at P4 and P10 (FIG. 11E). Their cardiomyocyte M-phase activity did not change (FIG. 11F). To determine the functional relationship between β-AR signaling and Ect2 functionally in generation of binucleated cardiomyocytes, we used siRNA (FIGS. 15A-15F) to knock down Ect2 in DKO cardiomyocytes. This experiment showed a significant increase in the percentage of binucleated cardiomyocytes generated (FIG. 11G, P=0.0032).

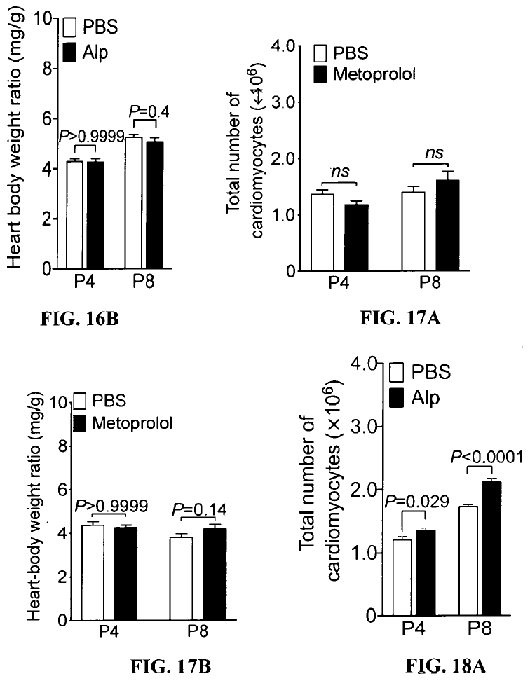

β-AR signaling regulates cardiomyocyte Ect2 expression, cytokinesis, and endowment: We explored pharmacological ways to control formation of binucleated cardiomyocytes and endowment growth. β-AR directly activates large heterotrimeric GTP-binding proteins (G proteins) of the stimulatory family (Gs). The natural compound forskolin (Fsk) mimics the active conformation of Gs. Accordingly, we added Fsk to cultured NRVMs, which decreased Ect2 mRNA levels (FIG. 12A). We administered forskolin in newborn mice and found a 37% increase in the proportion of binucleated cardiomyocytes after 4 days and a 21% increase after 8 days (FIG. 12B). We then administered propranolol, a blocker of β1- and β2-AR, in newborn mice. Propranolol decreased the proportion of binucleated cardiomyocytes by 21% after treatment from P1 to P4, and by 17% after treatment from P1 to P8 (FIG. 12C). This was associated with a 22% and 30% increase of cardiomyocyte endowment at P4 and P8, respectively (FIG. 12D), without a change in cardiomyocyte cell cycle entry (quantified by BrdU uptake, FIG. 12E) or progression to M-phase (quantified by H3P-staining, FIG. 12F) at P8. The heart weight was not changed (FIG. 16A). We examined the effect of another blocker of β₁- and β₂-AR, alprenolol. Administration of alprenolol from P1 to P8 decreased the formation of binucleated cardiomyocytes by 13% (FIG. 12G), corresponding to a 24% increase of cardiomyocyte endowment (FIG. 12H), but did not result in a change of the heart weight (FIG. 16B). These results show that reducing β-AR signaling by β-blocker administration in the neonatal phase enables cardiomyocyte abscission, thus increasing the endowment.

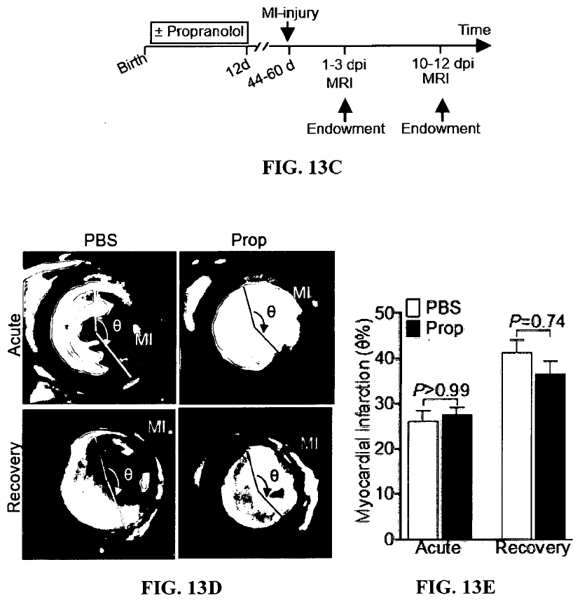

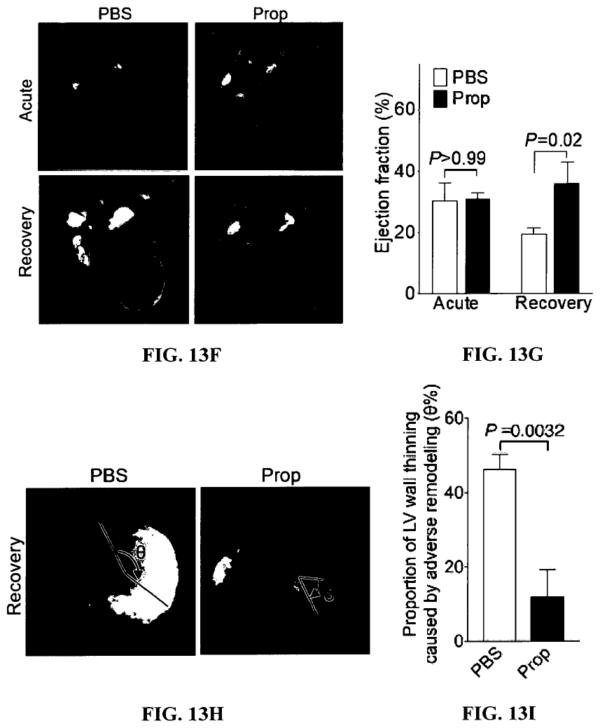

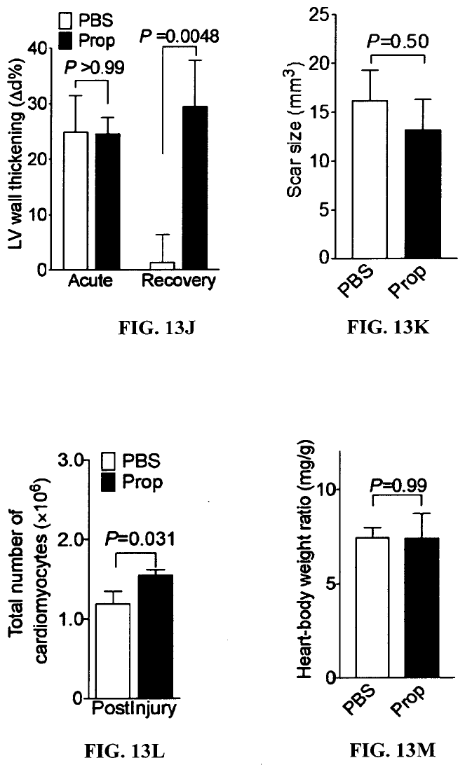

Neonatal propranolol-mediated increased endowment improves heart function and remodeling in adult mice after myocardial infarction: The increased cardiomyocyte endowment resulting from neonatal propranolol administration persisted until adulthood (FIG. 13A), but did not alter cardiac function (FIG. 13B). A larger endowment should confer a benefit after large-scale cardiomyocyte loss, for example, after myocardial infarction (MI). We tested this by administering propranolol in the neonatal period and then inducing myocardial infarction in adult mice (FIG. 13C). We determined cardiac structure and function with MRI (FIG. 13D). Two days after MI, control and propranolol-treated mice had the same infarct size as determined by late gadolinium-enhancement (LGE, FIGS. 13D, 13E) and ejection fraction (measured by MRI, FIGS. 13F, 13G). However, twelve days after MI, mice with propranolol-induced endowment growth had an MRI-measured ejection fraction of 42%, compared with 18% in control mice (FIGS. 13F, 13G). The thinned region of the LV myocardium after myocardial infarction was significantly smaller (FIG. 13H, 13I), and the relative systolic thickening was higher (FIG. 13J), indicating less adverse remodeling. Importantly, the region of myocardium affected by ischemia, visualized by LGE (FIGS. 13D, 13E), and the scar size, determined by histology (FIG. 13K), were not different. Propranolol-treated hearts had a 30% higher cardiomyocyte endowment after MI (determined by stereology, FIG. 13L), in keeping with the increased endowment before MI (see FIG. 13A). The heart weight was not changed (FIG. 13M), indicating that the higher endowment reduced the maladaptive hypertrophy, which drives adverse remodeling after MI. Taken together, these results demonstrate that rescuing cardiomyocyte cytokinesis failure with propranolol in development reduces adverse ventricular remodeling in adult mice.

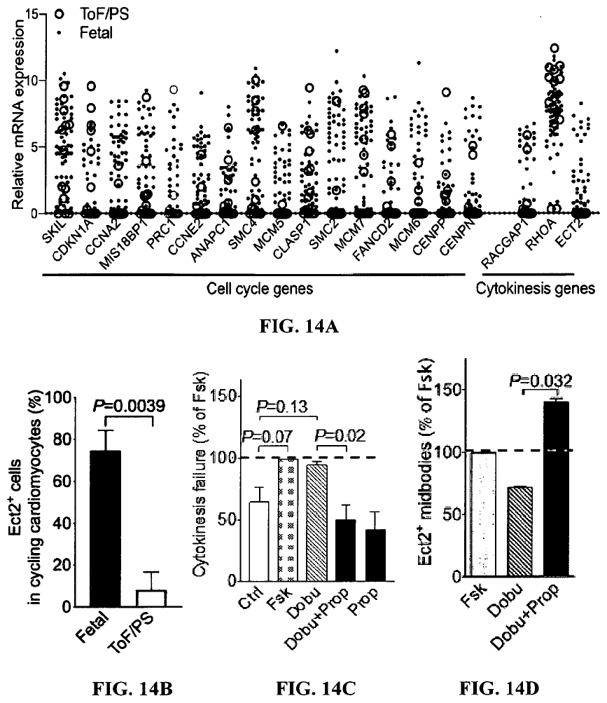

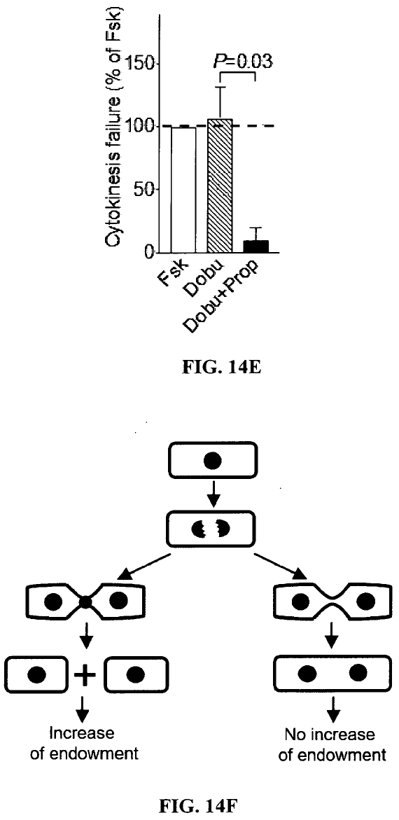

β-blockers rescue cytokinesis failure in cardiomyocytes from infants with ToF/PS: We determined to what extent the molecular mechanisms of cardiomyocyte cytokinesis failure we discovered are responsible for the increased proportion of binucleated cardiomyocytes in ToF/PS. To this end, we transcriptionally profiled single cardiomyocytes from infants with ToF/PS. Human control samples, corresponding in age and quality to the available freshly resected ToF/PS myocardium, are not available because infant hearts without disease are used for transplantation. As such, we turned to available human fetal hearts for comparative single cell transcriptional analysis. We normalized cardiomyocytes from human fetuses and ToF/PS infants together and imposed a rigorous quality control to ensure equal quality. Although expression of structural and functional genes may differ between fetuses and infants, we reasoned that expression of the cell cycle program, which is evolutionarily conserved, should be similar. In other words, we investigated we investigated whether single non-ToF/PS fetal and ToF/PS infant cardiomyocytes express cell cycle genes at the same amount when they enter the cell cycle. Indeed, the normalized mRNA expression levels of 16 cell cycle genes were similar between non-ToF/PS fetal and ToF/PS infant cardiomyocytes (FIG. 14A). We then compared the expression levels of the mRNAs encoding for Ect2's direct protein interaction partners in cytokinesis, RhoA and RacGAP1, which also showed similar mRNA expression levels. However, while fetal cardiomyocytes expressed Ect2, ToF/PS did not. We then identified cycling cardiomyocytes by the expression of cell cycle genes. The portion of cycling cardiomyocytes in ToF/PS infants that did not express Ect2 was >90% (FIG. 14B), corresponding to the proportion of cycling cardiomyocytes that go on to fail cytokinesis. This suggests that the majority of cycling cardiomyocytes in ToF/PS infants experience cytokinesis failure, which agrees with the results shown in FIGS. 2A-2E. In contrast, 75.6% of cycling human fetal cardiomyocytes expressed Ect2, indicating that the majority divided (FIG. 14B). This is consistent with the prior finding that human newborns have approximately 20-30% binucleated cardiomyocytes, which must be generated during fetal life. Thus, a large proportion of cycling cardiomyocytes in ToF/PS infants exhibits decreased Ect2 levels, similar to cardiomyocytes in neonatal mice (see FIG. 3B).

This finding prompted us to examine if regulating β-AR signaling would after cytokinesis failure in human cardiomyocytes. We used cultured human fetal cardiomyocytes and added Fsk, which maximally increased cardiomyocyte cytokinesis failure (FIG. 14C). We then treated human fetal cardiomyocytes with dobutamine to mimic the in vivo microenvironment of increased s-AR stimulation. Dobutamine increased binucleated cardiomyocytes to 95.2% of the Fsk-induced increase (FIG. 14C). Addition of propranolol blocked the dobutamine-stimulated increase in cardiomyocyte cytokinesis failure completely (FIG. 14C). We examined cardiomyocytes in cytokinesis by immunofluorescence microscopy, which showed that Ect2-positive midbodies were increased with propranolol (FIG. 14D). We then generated organotypic cultures of heart pieces from infants with ToF/PS and added BrdU to label cycling cardiomyocytes (FIG. 14E). Forskolin and dobutamine induced a maximal increase in the proportion of binucleated cardiomyocytes, and propranolol inhibited the dobutamine-stimulated increase completely (FIG. 14E). In conclusion, β-ARs regulate cytokinesis failure in cardiomyocytes from infants with ToF/PS and propranolol decreases this effect.

### DISCUSSION

To increase myocardial regeneration, conventional approaches stimulate cardiomyocyte proliferation in all phases of the cell cycle: cell cycle entry, progression to M-phase, and cytokinesis. Here, it is demonstrated that the number of cardiomyocytes can be increased simply by preventing cytokinesis failure. We show that β-ARs control the decision point of whether cardiomyocytes accomplish cytokinesis and divide, or fail and become binucleated (FIG. 14F). We therefore identified a function of β-AR signaling in regulating growth of cardiomyocyte endowment during development. Two major lines of evidence support this conclusion: 1. cardiomyocytes have decreased expression levels of the critical cytokinesis gene Ect2 when they become binucleated, and 2. blocking β-AR signaling disinhibits Ect2 transcription, which increases division in cycling cardiomyocytes and increases their numbers (endowment). We show that the higher endowment confers benefit after MI in adult mice and that the molecular mechanisms are also operative in human cardiomyocytes.

By identifying that β-ARs regulate Ect2 gene transcription, we were able to conduct experiments demonstrating that cardiomyocyte cytokinesis can be manipulated without altering cell cycle entry or progression. First, Ect2 gene inactivation or expression of the Ect2 cDNA altered cardiomyocyte cytokinesis but did not change cell cycle entry or progression. Second, increasing Ect2 gene transcription with β-AR gene knockout or β-blockers also failed to alter cardiomyocyte cell cycle entry or progression. Prior studies examining the mechanisms of formation of binucleated cardiomyocytes did not distinguish cytokinesis failure from regulation of the other phases of the cell cycle. Consequently, our method and use represent an important advance.

Our results imply that cytokinesis failure stops further cardiomyocyte division. To test this hypothesis, we performed a back-of-the-envelope calculation of cardiomyocytes predicted to be generated due to propranolol administration in mice from P4 to P8, for which initial and final data are available. Between P4 and P8, the cardiomyocyte endowment in propranolol-treated pups increased by 0.35×10⁶ and in PBS-treated pups by 0.17×10⁶ cardiomyocytes. Thus, the propranolol-induced increase between P4 and P8 was 0.18×10⁶ cardiomyocytes. How does this compare to decreased cytokinesis failure, calculated from the decrease of the percentage of binucleated cardiomyocytes in propranolol-treated pups? At P8, propranolol-treated pups had 13.6% fewer cardiomyocytes undergoing cytokinesis failure than PBS-treated pups. Taking the endowment of PBS-treated pups at P4 (1.69×10⁶ cardiomyocytes), this corresponds to 0.22×10⁶ additional cardiomyocytes generated in propranolol-treated pups at P8. Thus, the number of cardiomyocytes predicted to be generated by enabling completion of cytokinesis with propranolol administration (0.22×10⁶) corresponds to the number of cardiomyocytes calculated from the counts (0.18×10⁶).

We show that cardiomyocyte cytokinesis failure is significant for patients with ToF/PS, where our results predict that it reduces the cardiomyocyte endowment by 25%. These changes develop in the first 6 months after birth, before any surgical intervention, and persist in older ToF/PS patients.

Based on the results presented herein, β-blockers could turn cytokinesis failure to increased cardiomyocyte division in infants with ToF/PS. The results in mice demonstrate that promoting the progression of cytokinesis to abscission in the postnatal period Increases the endowment, which improves remodeling after myocardial infarction. This suggests that formation of a higher or lower cardiomyocyte endowment during development connects to outcomes in adult human patients. This could be tested with β-blocker administration in human infants with ToF/PS to increase the endowment, followed by measuring clinical outcomes, such as myocardial function and risk of heart failure development β-blockers have been used sporadically to treat and prevent cyanotic spells in ToF/PS. Our results predict that administration of β-blockers should produce the largest effect on cardiomyocyte cytokinesis during the first 6 months after birth.

### Example 2

### Materials and Methods

Metoprolol was dissolved in PBS (1 μg/μl). The metoprolol was administered to neonatal mice using retro-orbital injection twice a day at the dose of 10 μl solution each 1 g body weight In the 4-day-old group (P4), the drug administration was performed at 9 am and 3 μm, respectively, from P1 and P3, then only injections at 9 am were performed at P4. The hearts were isolated at P4 in the afternoon. In the 8-day-old group (P8), the drug administration was performed at 9 am and 3 μm, respectively, from P1 and P7, then only injections at 9 am were performed at P8. The hearts were isolated at P8 in the afternoon.

### Results and Discussion

The resulting total number of cardiomyocytes and heart-body weight ratio can be found in FIGS. 17A and 17B, respectively. Metoprolol treatment did not alter the total number of heart muscle cells and the heart-body-weight ratio in neonatal mice. Because Metoprolol is a β₁-selective blocker, β₂-selective and non-specific beta blockers may be preferable for increasing cardiomyocyte endowment. However, each beta blocker has different therapeutic windows and activity, and we cannot rule out efficacy based on this single data point. Beta blockers with different selectivity can overlap their respective selectivity at higher concentrations, and as such, for example, metoprolol may be administered to achieve higher systemic concentrations,

### Example 3

### Materials and Methods

Alprenolol was dissolved in PBS (1 μg/μl). The alprenolol was administered to neonatal mice using retro-orbital injection twice a day at the dose of 10 μl solution each 1 g body weight In the 4-day-old group (P4), the drug administration was performed at 9 am and 3 pm, respectively, from P1 and P3, then only injections at 9 am were performed at P4. The hearts were isolated at P4 in the afternoon. In the 8-day-old group (P8), the drug administration was performed at 9 am and 3 pm, respectively, from P1 and P7, then only injections at 9 am were performed at P8. The hearts were isolated at P8 in the afternoon.

### Results and Discussion

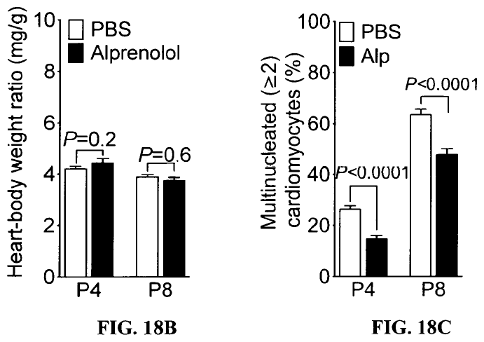

The resulting total number of cardiomyocytes, heart-body weight ratio, and percentage of multinucleated cardiomyocytes can be found in FIGS. 18A-18C, respectively. Alprenolol treatment increases the total number of heart muscle cells without altering the heart-body-weight ratio in neonatal mice.

### Example 4

### Exemplary Clinical Trial

β-blockers could increase cardiomyocyte proliferation in ToF/PS, and with that, strengthen the heart. β-blockers have an established place in adult patients, including for pulmonary artery hypertension (PAH). Although extensively used, very few controlled clinical trials of β-blockers have been performed in CHD.

Objective: As primary outcome, cardiomyocyte proliferation will be determined using the innovative ¹⁵N-thymidine labeling approach with MIMS readout to develop a detailed timeline of the propranolol-induced reactivation of cardiomyocyte proliferation.

Study Design: A randomized, controlled, double-blinded, single-center trial of propranolol will be administered to ToF/PS infants to determine if β-blocker administration in increases cardiomyocyte division. Preliminary results in mice indicate that propranolol administration will be most effective between birth and 6 months of age.

A ToF/PS infant patient having a heart defect characteristic of tetraology of the fallot will be administered ¹⁵N-thymidine (50 mg/kg p.o.) on 5 consecutive days. Propranolol will be administered to the ToF/PS infant patient, until the defect is corrected. Heart structure and function of the infant patient will be determined using a cardiac MRI and echocardiography. The heart function of the infant patient will be followed for several years to determine possible improvements of RV remodeling, failure, and arrhythmia, which is feasible because most patients retum for long-term care. The effect of the propranolol administration on cardiomyocyte proliferation will be determined by analyzing ¹⁵N-thymidine uptake in cardiomyocytes.

Having described this invention above, it will be understood to those of ordinary skill in the art that the same can be performed within a wide and equivalent range of conditions, formulations and other parameters without affecting the scope of the invention or any embodiment thereof. Any document incorporated herein by reference is only done so to the extent of its technical disclosure and to the extent it is consistent with the present document and the disclosure provided herein.

1. A method of treating patients less than 6 months past term having a congenital heart defect and a reduced cardiomyocyte endowment resulting from heart cell division failure, comprising administering to the patient a nonspecific beta-blocker, a β₁-beta blocker, a β₂-beta blocker, or a combination thereof, in an amount and for a duration effective to induce cardiomyocyte cytokinesis in the patient and expansion of the cardiomyocyte endowment in the patient, thereby reducing a percentage of binucleated cells in heart tissue in the patient, increasing cardiomyocyte endowment by at least 5% in the patient, and/or improving heart function and resilience to heart injury in the patient.

2. The method of claim 1, further comprising determining a percentage of binucleated cardiomyocytes in heart tissue of the patient or determining a presence of multinucleated cardiomyocytes in heart tissue of the patient at one or more times prior to or during administration of the beta-blocker to the patient.

3. The method of claim 2, further comprising determining a percentage of binucleated cardiomyocytes in heart tissue of the patient at two or more time points including a time point during or after administration of the beta blocker to the patient, and determining if the percentage of binucleated cardiomyocytes in the heart tissue is decreased, indicating expansion of the cardiomyocyte endowment in the patient.

4. The method of claim 1, comprising determining heart tissue growth or cardiac mass in the patient to determine an increase in cardiomyocyte endowment in the patient.

5. The method of claim 1, wherein the congenital heart defect results in above right ventricle systolic pressure (RVSP), further comprising determining RVSP at one or more time points during treatment of the patient with the beta blocker.

6. The method of claim 1, comprising discontinuing administration of the beta blocker after determining that the binucleated cardiomyocyte percentage in heart tissue in the patient is normalized and/or cardiomyocyte endowment is increased at least 5% in the patient.

7. The method of claim 1, wherein the patient is non-cyanotic or non-hypoxic.

8. The method of claim 1, wherein the beta blocker is a nonspecific beta-blocker.

9. The method of claim 1, wherein the beta blocker is a β₂ beta-blocker.

10. The method of claim 1, wherein the beta-blocker comprises propranolol or alprenolol.

11. The method of claim 1, wherein the congenital heart defect is a defect associated with tetralogy of Fallot.

12. The method of claim 1, wherein the patient has a hypoplastic or absent conal septum, stenosis of the left pulmonary artery, a bicuspid pulmonary valve, a right-sided aortic arch, coronary artery anomalies, a patent foramen ovale or atrial septal defect, an atrioventricular septal defect, a partial or complete pulmonary vein return anomaly, and/or pulmonary atresa.

13. The method claim 1, wherein the congenital heart defect is, or is a defect associated with: trilogy of Fallot; aortic valve stenosis; coarctation of the aorta; Ebstein's anomaly; patent ductus arteriosus; pulmonary valve stenosis; septal defect, such as an atrial septal defect or an ventricular septal defect; a single ventricle defect, such as hypoplastic left heart syndrome or tricuspid atresia; total or partial anomalous pulmonary venous connection (TAPVC); transposition of the great arteries; or truncus arteriosus.

14. The method of claim 1, wherein the congenital heart defect is an anterior malalignment of the infundibular septum with the muscular septum.

15. The method of claim 1, wherein the congenital heart defect is one or more of pulmonary valve stenosis, a ventricular septal defect, an overriding aorta, and right ventricular hypertrophy.

16. The method of claim 1, wherein the patient has undergone surgery to repair one or more defects resulting from the congenital heart disease in the patient, and the beta blocker is administered to the patient continuously for at least two weeks, or for at least one month after the surgery to increase cardiomyocyte endowment in the patient.

17. The method of claim 1, wherein treatment of the patient with the beta blocker is initiated prior to closure of the foramen ovale in a patient not having a patent foramen ovale or ductus arteriosus, in a patient not having patent ductus arteriosus.

18. The method of claim 1, wherein the patient is human.

19. The method of claim 1, to lower risk of complications relating to myocardial infarction in the patient, such as heart failure.

20. The method of claim 1, further comprising administering one or more additional therapeutic agents to the patient during treatment of the patient with the beta blocker.

21. The method of claim 20, wherein the one or more additional therapeutic agents is a cell growth factor or mitogen in an amount effective to stimulate cardiomyocyte cell growth or expansion in the patient.

22. The method of claim 21, wherein the cell growth factor or mitogen is periostin, neuregulin, a fibroblast growth factor, or NRG61 (SEQ ID NO: 2).

23-47. (canceled)


SEQUENCE LISTING (29 sequences)

SEQ ID NO: 1 - protein, 23 aa, Artificial Sequence
  Neuregulin
AEKEKTFCVNGGECFMVKDLSNP

SEQ ID NO: 2 - protein, 61 aa, Artificial Sequence
  NRG61
SHLVKCAEKEKTFCVNGGECFMVKDLSNPSRYLCKCPNEFTGDRCQNYVMASFYKAEELYQ

SEQ ID NO: 3 - protein, 53 aa, Artificial Sequence
  Neuregulin peptide EGF53
SHLVKCAEKEKTFCVNGGECFMVKDLSNPSRYLCKCPNEFTGDRCQNYVMASF

SEQ ID NO: 4 - protein, 55 aa, Artificial Sequence
  Neuregulin peptide NRG55
SHLVKCAEKEKTFCVNGGECFMVKDLSNPSRYLCKCPNEFTGDRCQNYVMASFYK

SEQ ID NO: 5 - protein, 57 aa, Artificial Sequence
  Neuregulin peptide NRG57
SHLVKCAEKEKTFCVNGGECFMVKDLSNPSRYLCKCPNEFTGDRCQNYVMASFYKAE

SEQ ID NO: 6 - protein, 59 aa, Artificial Sequence
  Neuregulin peptide NRG59
SHLVKCAEKEKTFCVNGGECFMVKDLSNPSRYLCKCPNEFTGDRCQNYVMASFYKAEEL

SEQ ID NO: 7 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 8 - DNA, 22 bp, Artificial Sequence
  Ect2 primer forward
agaaggtgctggacatccgaga

SEQ ID NO: 9 - DNA, 22 bp, Artificial Sequence
  Ect2 primer reverse
cctttggaagctcctctgacgt

SEQ ID NO: 10 - DNA, 23 bp, Artificial Sequence
  beta-actin primer forward
cattgctgacaggatgcagaagg

SEQ ID NO: 11 - DNA, 22 bp, Artificial Sequence
  beta-actin primer reverse
tgctggaaggtggacagtgagg

SEQ ID NO: 12 - DNA, 23 bp, Artificial Sequence
  GAPDH primer forward
catcactgccacccagaagactg

SEQ ID NO: 13 - DNA, 23 bp, Artificial Sequence
  GAPDH primer reverse
atgccagtgagcttcccgttcag

SEQ ID NO: 14 - DNA, 21 bp, Artificial Sequence
  Ect2 primer forward
cgcaaggagaaaagtttaggg

SEQ ID NO: 15 - DNA, 20 bp, Artificial Sequence
  Ect2 primer reverse
catcctggcctcataattgg

SEQ ID NO: 16 - DNA, 24 bp, Artificial Sequence
  Racgap1 primer forward
ttcaaaatgaagaaacgagaaaag

SEQ ID NO: 17 - DNA, 19 bp, Artificial Sequence
  Racgap1 primer reverse
agggccatcgatgaactgt

SEQ ID NO: 18 - DNA, 21 bp, Artificial Sequence
  MKLP1 primer forward
tggaccgatgtagaaacaagg

SEQ ID NO: 19 - DNA, 18 bp, Artificial Sequence
  MKLP1 primer reverse
gttgtgcgtggtgctgat

SEQ ID NO: 20 - DNA, 19 bp, Artificial Sequence
  GEF-H1 primer forward
cggatcgaatccctcactc

SEQ ID NO: 21 - DNA, 19 bp, Artificial Sequence
  GEF-H1 primer reverse
tctccttttcccgggtctt

SEQ ID NO: 22 - DNA, 19 bp, Artificial Sequence
  beta-actin primer forward
cccgcgagtacaaccttct

SEQ ID NO: 23 - DNA, 18 bp, Artificial Sequence
  beta-actin primer reverse
cgtcatccatggcgaact

SEQ ID NO: 24 - DNA, 20 bp, Artificial Sequence
  GAPDH primer forward
ggcaagttcaacggcacagt

SEQ ID NO: 25 - DNA, 22 bp, Artificial Sequence
  GAPDH primer reverse
tggtgaagacgccagtagactc

SEQ ID NO: 26 - DNA, 20 bp, Artificial Sequence
  Rosa26 primer forward
cgtttccgacttgagttgcc

SEQ ID NO: 27 - DNA, 20 bp, Artificial Sequence
  Rosa26 primer reverse
actcgggtgagcatgtcttt

SEQ ID NO: 28 - DNA, 19 bp, Artificial Sequence
  Ect2flox primer forward
tcctccgggtggaccagag

SEQ ID NO: 29 - DNA, 21 bp, Artificial Sequence
  Ect2flox primer reverse
ctggcttcataattggagtgc